(19) United States
(12) Patent Application Publication
(10) Pub. No.: US 2015/0079105 A1
(43) Pub. Date: Mar. 19, 2015

(54) TREATMENT OF ACUTE INFLAMMATION IN THE RESPIRATORY TRACT

(71) Applicant: UCL Business PLC, London (GB)

(72) Inventors: Rachel Chambers, London (GB); Paul Mercer, London (GB); Andrew Williams, London (GB)

(73) Assignee: ucl Business PLC, London (GB)

(21) Appl. No.: 14/389,718

(22) PCT Filed: Mar. 15, 2013
(86) PCT No.: PCT/GB2013/050665

§ 371 Date: Sep. 30, 2014

(30) Foreign Application Priority Data
Mar. 30, 2012 (GB) 1205739.4

(51) Int. Cl. A61K 39/395; A61K 38/00; A61K 31/7105; C07K 16/24; C12N 15/113

(52) U.S. Cl. CPC A61K 39/3955; C07K 16/24; C12N 15/1136; A61K 31/7105; A61K 38/00; A61K 2039/505
USPC 424/145.1; 530/388.1; 536/23.1; 514/44 R; 536/24.5; 514/44 A; 514/1.5

(57) ABSTRACT

This invention relates to the field of molecular physiology. Specifically, this invention relates to the prevention and/or treatment of acute inflammation of the respiratory tract, especially acute lung injury (ALI) or acute respiratory distress syndrome (ARDS). Levels of CCL7 have been demonstrated to be increased in patients suffering from such conditions and animal models of such conditions. Antagonists of CCL7 and/or other members of the PAR1-CCL7 axis, or CCL2 can be used to prevent and/or treat these conditions.

### FIELD OF THE INVENTION

The invention is in the field of molecular physiology and relates to the use of antagonists of CCL7, PAR₁, other members of the CCL7-PAR₁ axis, or CCL2 for use in the treatment or prevention of acute inflammation associated with the accumulation of neutrophils in the respiratory tract, in particular acute lung injury (ALI) and acute respiratory distress syndrome (ARDS).

### BACKGROUND OF THE INVENTION

Acute lung injury (ALI) and its more severe form acute respiratory distress syndrome (ARDS) arise from a variety of local and systemic insults, including pneumonia, trauma and sepsis, which can all lead to acute respiratory failure. ALI/ARDS are common, life-threatening conditions that affect 79/100,000 people in the UK each year, with a mortality rate of 30-60% (Monchi, M. et al. Am. J. Respir. Crit Care Med. 158, 1076-1081 (1998)). The early stages of ALI and ARDS are associated with an influx of neutrophils into the injured tissue (Abraham, E. Crit Care Med. 31, S195-S199 (2003)). In addition to neutrophilic inflammation, these conditions are characterized by diffuse alveolar damage, disruption of the alveolar capillary barrier and pulmonary oedema. Although a rapid innate immune response provides immediate host protection against infectious microorganisms, excessive neutrophil accumulation can lead to exuberant inflammation and severe tissue damage (Grommes, J. & Soehnlein, O. Mol. Med. 17, 293-307 (2011)). Despite some improvements using lung-protective ventilator strategies, both morbidity and mortality remain high, as respiratory collapse and multiple organ failure occur in up to 40% of patients with ALI/ARDS (Marshall, R. P. et al. Am. J. Respir. Crit Care Med. 162, 1783-1788 (2000)).

The proteinase activated receptor 1 (PAR₁) belongs to a family of seven-transmembrane G protein-coupled receptors that are activated by the proteolytic unmasking of a tethered ligand (Vu, T. K. et al. Cell 64, 1057-1068 (1991)). Evidence obtained from biochemical studies and from PAR₁-knockout mice suggest a key role for the major high-affinity thrombin receptor PAR₁ in mediating the complex interplay between coagulation and inflammation in lung disease (Howell, D. C. et al. Am. J. Pathol. 166, 1353-1365 (2005); Jenkins, R. G. et al. J. Clin. Invest 116, 1606-1614 (2006); Scotton, C. J. et al. J. Clin. Invest 119, 2550-2563 (2009) and Chambers, R. C. Br. J. Pharmacol. 153 Suppl 1, S367-S378 (2008)).

Activation of PAR₁ on epithelial cells, monocytes/macrophages and vascular endothelial cells leads to the release of several proinflammatory mediators and exerts differential, concentration dependent effects on endothelial barrier function. Elevated chemokine levels and the upregulation of cell-adhesion molecules on the endothelium in response to PAR₁ activation facilitates the recruitment of inflammatory cells from the circulation and into the lungs (Chambers, R. C. Eur. Respir. J. Suppl 44, 33 s-35 s (2003)). However, it is uncertain as to what extent the release of PAR₁-mediated pro-inflammatory cytokines and chemokines contributes to neutrophilic inflammation in ALI/ARDS.

There is therefore a need to identify alternative therapeutic targets and novel drugs which, in isolation or in combination with existing therapies, may be more effective and possess fewer off-target effects for the treatment of ALI/ARDS.

### SUMMARY OF THE INVENTION

We have identified CCL7 (CCL7 gene identifiers: HGNC: 10634; Ensemb1: ENSG00000108688 (Ensembl version ENSG00000108688.7); UniProtKB (version 125): P80098) as a drugable target for the treatment and/or prevention of ALI/ARDS. CCL7 (monocyte chemoattractant protein-3, MCP-3) is a member of the CC-chemokine family (β-chemokines) characterized by two adjacent cysteine residues at the amino terminal of the mature protein. In general, CC-chemokines are small molecules of approximately 8-12 kDa, which perform several important functions during the orchestration of an immune response. They are capable of forming a chemotactic gradient that attracts various leukocytes towards the site of production, can contribute to the activation of certain cell types and are involved in diverse effector functions such as degranulation, gene expression and cell motility. Like most chemokines, CC-chemokines have pleiotropic functions depending on the tissue and cellular source and the context in which they are expressed among the milieu of other chemokines and cytokines. CCL7 is no exception in that it is expressed by several cell types including macrophages, dendritic cells (DCs) and epithelial cells. Although once thought to be a specific macrophage chemoattractant, it is now clear that CCL7 exerts effects on monocytes, macrophages, DCs, T cells, NK cells, neutrophils, eosinophils, basophils and mast cells, making it the most promiscuous of all CC-chemokines and in so doing influencing the pathogenesis of several important diseases including, along with CXCL10, asthma (Michalec L. et al, J. Immunol. 168, 846-852 (2002).

To date, CCL7 has however not been implicated in ALI/ARDS. Instead, CXCL8 (IL-8) and the rodent homologues CXCL1 (KC) and CXCL2 (MIP-2) are thought to be central to neutrophil recruitment into the lung during ALI. Important correlations have been made, in clinical ALI samples, between increased IL-8 and neutrophil migration into the airspaces (Miller, E. J. et al Crit Care Med, 24, 1448-1454 (1996)) and with mortality (Miller, E. J. et al Am Rev Respir Dis, 146, 427-432 (1992)). Further, only IL-8 consistently correlates with the number of neutrophils and severity of disease (Goodman, R. B. et al Cytokine Growth Factor Rev, 14, 523-535 (2003) and Villard, J. et al Am J Respir Crit Care Med, 152, 1549-1554 (1995)) and is therefore considered the main neutrophil chemoattractant in ALI/ARDS.

The present inventors have however demonstrated that PAR₁ signalling mediates CCL7 expression, that acute neutrophilic inflammation is dependent on CCL7 and that CCL7 regulates the chemotaxis of human neutrophils during ALI. The present inventors have also shown that neutrophils from the lungs of LPS challenged mice express increased CCR1 and CCR2, but decreased expression of CXCR2 molecules. Therefore, the inventors have shown that neutrophils can respond to CC chemokines.

The present inventors have further demonstrated that PAR₁ signalling mediates the expression of the related chemokine CCL2 (also known as MCP-1) and that acute neutrophilic inflammation is dependent on CCL2. The present inventors have shown that neutrophils form the lungs of LPS challenged mice express the CCL2 receptor CCR2.

Therefore, the present inventors have identified CCL2 (CCL2 gene identifiers: HGNC: 10618; Ensembl: ENSG00000108691 (Ensembl version ENSG00000108691.4); UniProtKB (version 167): P13500) as a drugable target for the treatment and/or prevention of ALI/ARDS.

Thus, the present inventors have now identified CCL7 and CCL2 as drugable targets for the treatment and/or prevention of acute inflammation associated with the accumulation of neutrophils in the respiratory tract, in particular in ALI/ARDS. The findings in relation to PAR₁ and CCL7 raise the possibility of targeting PAR₁ and/or other members of the PAR₁-CCL7 axis for the same purpose.

Accordingly, the present invention provides an antagonist of CCL7, PAR₁, another member of the PAR₁-CCL7 axis, or CCL2 for use in the treatment or prevention of acute inflammation associated with the accumulation of neutrophils in the respiratory tract.

The invention also provides the use of an antagonist of CCL7, PAR₁, another member of the PAR₁-CCL7 axis, or CCL2 in the manufacture of a medicament for the treatment or prevention of acute inflammation associated with the accumulation of neutrophils in the respiratory tract.

The invention also provides a method of treating or preventing acute inflammation associated with the accumulation of neutrophils in the respiratory tract comprising administering to a patient in need thereof an effective amount of an antagonist of CCL7, PAR₁, another member of the PAR₁-CCL7 axis, or CCL2.

### BRIEF DESCRIPTION OF THE DRAWINGS

FIG. 1. Mice were killed 3 hours after LPS (125 μg/kg i.n.) or saline challenge with and without the PAR₁ antagonist RW7-58259 (5 mg/kg) dosed therapeutically (i.p) after 30 min. Lungs were lavaged (1.5 ml PBS total) or removed and homogenised for FACS analysis. Total (A) and differential BAL fluid neutrophils (B) were quantified by haemocytometer counts of cytospins. Neutrophil myeloperoxidase (MPO) activity in lung homogenates was assessed by ELISA (C). Gr-1+ neutrophils (Gr-1^{high} F4/80^{neg}) isolated from BAL fluid (D) or lung homogenates (E) were further assessed by flow cytometry. BALF macrophages were assessed by cytospin analysis (E). Alveolar leak was measured by serum albumin levels recovered from BALF (F). Panel shows mean values for n=5/group from three separate experiments. Data were analysed by one way ANOVA with Neuman-Keuls Post Hoc test: ***p<0.0001, **p<0.01, *p<0.05.

FIG. 2. Mice were killed three hours after LPS (125 ng/kg i.n.) challenge with and without the specific PAR₁ antagonist SCH530348 (10 mg/kg) dosed therapeutically i.p. immediately after LPS challenge. Lungs were lavaged and total cells (A) and neutrophils (B) counted using a haemocytometer and cytospin preparation. Whole lung was removed and homogenised. The chemokines CXCL1 (KC) and CCL7 were measured by ELISA (C and D). Data were analysed by one way ANOVA with Neuman-Keuls Post Hoc test: *p<0.05.

FIG. 3. Mice were killed 6 h or 24 h after LPS (125 μg/kg i.n.) or saline challenge with and without the PAR₁ antagonist RWJ-58259 (5 mg/kg) dosed therapeutically (i.p) after 30 min. Lungs were lavaged (1.5 ml PBS total). Data were analysed by one way ANOVA with Neuman-Keuls Post Hoc test.

FIG. 4. Mice were killed three hours after inoculation with S. pneumoniae (serotype 19, 50 μl/mouse, 5×10⁶ CFU/mouse i.n.) with and without the PAR₁ antagonist RWJ-58259 (5 mg/kg) dosed therapeutically i.p. after 30 min. Lungs were lavaged (1.5 ml PBS total) and total BAL fluid leukocytes (a) and neutrophils (b) were quantified. S. pneumoniae was recovered from lung homogenates and individual colonies counted (c). Panels show mean values for n=5/group from two separate experiments. Data were analysed by one way ANOVA with Neuman-Keuls Post Hoc test: ***p<0.0001, *p<0.05.

FIG. 5. Mice were killed three hours after inoculation with S. pneumoniae (serotype 2, clinical isolate D39, 50 μl/mouse, 5×10⁶ CFU/mouse i.n.) with and without the PAR₁ antagonist RWJ-58259 (5 mg/kg) dosed therapeutically i.p. after 30 min. Lungs were lavaged (1.5 ml PBS total) and total BAL fluid leukocytes (A), macrophages (B) and neutrophils (C) were quantified. Bronchoalveolar lavage fluid was collected and levels of thrombin-anti-thrombin (TAT) and serum albumin were quantified by ELISA (D and E). Panels show mean values for n=5/group from two separate experiments. Data were analysed by one way ANOVA with Neuman-Keuls Post Hoc test: ***p<0.0001, *p<0.05.

FIG. 6. Mice were killed three hours after inoculation with S. pneumoniae (serotype 2, clinical isolate D39, 50 μl/mouse, 5×10⁶ CFU/mouse i.n.) with and without the PAR₁ antagonist RWJ-58259 (5 mg/kg) dosed therapeutically i.p. after 30 min. Lungs were lavaged (1.5 ml PBS total) and bacterial colony forming units (cfu) counted after 3 h (A) and 24 h (B). Bacterial invasive disease was measured by cfu in the lung (C) and spleen (D) after 24 h. Data were analysed by one way ANOVA with Neuman-Keuls Post Hoc test: n.s. not significant.

FIG. 7. Mice were killed 3 hours after LPS (125 μg/kg i.n.) or saline challenge with or without the the highly selective PAR₁ antagonist RWJ-58259 (5 mg/kg) dosed therapeutically (i.p.) after 30 min. Lungs were removed, snap frozen and homogenised under liquid nitrogen before RNA was isolated and run on a low density gene array consisting of 151 inflammatory markers (A). Gene expression following LPS treatment revealed 51 markers to be differentially regulated (B). A further 25 markers exhibited reduced expression following PAR₁ antagonism (C). Of these genes the pro-inflammatory cytokines TNF and IL-6, and the neutrophil chemoattractants CXCL1 and CXCL2, in addition to the chemokines CCL2 and CCL7 are further depicted (D). Data were analysed by one way ANOVA with Newman-Keuls Post Hoc test:, **p<0.01, *p<0.05.

FIG. 8. Mice were killed three hours after LPS (125 μg/kg i.n.) or saline challenge with or without the PAR₁ antagonist RWJ-58259 (5 mg/kg) dosed i.p. after 30 min. Lungs were removed and homogenized. Levels of TNF (A), IL-6 (B) CXCL1 (C), and CXCL2 (D) protein were measured in lung homogenates using a Luminex bead array. Protein levels of CCL2 (H) and CCL7 (I) were measured from lung homogenates by ELISA. Mean and sem values of n=5 per group are shown. Data were analysed by one way ANOVA with Newman-Keuls post hoc test: ***p<0.001, **p<0.01, *p<0.05.

FIG. 9. Mice were killed 6 h or 24 h after LPS (125 μg/kg i.n.) or saline challenge with and without the PAR₁ antagonist RWJ-58259 (5 mg/kg) dosed therapeutically (i.p) after 30 min. Lungs were removed and homogenized. Table shows the profile of 151 inflammatory mediators analysed using a low density array of the lung homogenates.

FIG. 10. Mice were killed three hours after LPS (125 μg/kg i.n.) or saline challenge. Mice were administered CCL7 neutralizing antibody (10 μg/mouse) within the nasal challenge volume. Lungs were lavaged (1.5 ml PBS total) and differential BAL fluid neutrophils quantified following administration of anti-CCL7 (a). CCL7 (b) chemokine levels were measured in lung homogenates from treated mice by ELISA. Mean and sem values of n=5 per group for 2 separate experiments are shown for each treatment. Data were analysed by one way ANOVA with Neuman Keuls post hoc test: ***p<0.0001, **p<0.01, *p<0.05. Mice were treated with CCR2 specific blocking antibody (anti-CCR2; MC21) or isotype control (MC67) (10 μg/mouse i.p.) 12 hours prior to LPS (125 μg/kg i.n.) or saline challenge. Lungs were lavaged (1.5 ml PBS total) and differential BAL fluid neutrophils quantified (c). Gr-1+/CD11b+ monocytes in blood were quantified by FACS (d, circled). Mice were further treated with a CCR1/CCR2 antagonist 12 hours prior to LPS or saline challenge (e). Mean and sem values of n=6 per group are shown. Data were analysed by one way ANOVA with Neuman Keuls post hoc test: ***p<0.0001, **p<0.01.

FIG. 11. CC-chemokines influence early leukocyte accumulation in response to LPS challenge. Mice were killed three hours after LPS (125 μg/kg i.n.) or saline challenge. Mice were administered with anti-CCL2 or anti-CCL7 neutralizing antibody (10 μg/mouse), or control IgG, within the nasal challenge volume. Lungs were removed, homogenised and CCL2 levels measured by ELISA (A) following anti-CCL2 antibody treatment. Lungs were lavaged and BAL fluid total cells (B) and neutrophils (C) quantified following administration of anti-CCL2. In addition, lungs were removed, homogenised and CCL7 levels measured by ELISA (D) following anti-CCL7 antibody treatment. Lungs were lavaged and BAL fluid total cells (E) and neutrophils (F) quantified. Mean and sem values of at least n=5 per group for 2 separate experiments are shown for each treatment. Data were analysed by one way ANOVA with Newman-Keuls post hoc test: **p<0.01, *p<0.05; n.s. not significant.

FIG. 12. Mice were killed three hours after LPS (125 μg/kg i.n.) challenge. Mice were administered CXCL10, CX3CR1 or CCL12 neutralizing antibody (10 μg/mouse) within the nasal challenge volume. Lungs were lavaged (1.5 ml PBS total) and differential BAL fluid neutrophils quantified following administration of anti-CXCL10, anti-CX3CR1 or anti-CCL12 neutralizing antibodies. CXCL10 (a), CX3CR1 (b) or CCL12 (c) chemokine levels were measured in lung homogenates from treated mice by ELISA. Data were analysed by one way ANOVA with Neuman Keuls post hoc test.

FIG. 13. Mice were killed three hours after inoculation with S. pneumoniae (serotype 2, clinical isolate D39, 50 μl/mouse, 5×10⁶ CFU/mouse i.n.) with and without specific neutralizing antibody to CCL7 (10 μg/mouse i.n. within challenge volume). Lungs were lavaged (1.5 ml PBS total) and total BAL fluid leukocytes (A), and neutrophils (B) were quantified. Bacteria (cfu) recovered from the BALF were also counted (C). Data were analysed by one way ANOVA with Neuman-Keuls Post Hoc test: **p<0.001, *p<0.05.

FIG. 14. Mice were killed three hours after LPS (125 μg/kg i.n.) challenge with or without PAR₁ antagonist. LDA analysis of CXCL10 (A) and CX3CL1 (B) mRNA levels (normalized to 18 s housekeeping gene). Mice were administered CXCL10 or CX3CL1 neutralizing antibody (10 μg/mouse) within the LPS nasal challenge volume. Lungs were lavaged (1.5 ml PBS total) and differential BAL fluid neutrophils quantified following administration of anti-CXCL10 (C) or anti-CX3CL1 (D) neutralizing antibodies. Data were analysed by one way ANOVA with Neuman Keuls post hoc test: **p<0.01.

FIG. 15. Naïve mice were administered with either rCCL2 or rCCL7 (500 ng/mouse, i.n.) and 3 h later BAL fluid was recovered. BAL fluid total cell counts (A) and total neutrophils (B) were calculated from differential cell counts performed on cytospin preparations. The percentage of neutrophils in BAL fluid was also calculated (C). Differential cell counts were performed on cytospin preparations following saline (D), rCCL2 (E) or rCCL7 (F) administration. Data were analysed by one way ANOVA with Newman-Keuls post hoc test: *p<0.05, **p<0.01,*** p<0.001. Arrows indicate: N, neutrophil; M, monocyte/macrophage.

FIG. 16. Mice were challenged with 125 μg/kg LPS (i.n.) and whole lungs inflated and fixed after 3 h. Immunohistochemical staining of CCL7 (a, b an c) or Gr-1 (d, e and f) was compared between saline treated controls and LPS challenged mice with or without treatment with the PAR₁ antagonist RWJ-58259 (5 mg/kg) dosed therapeutically after 30 min. [AEW]. Endothelial-epithelial barrier disruption was measured by ELISA as the amount of serum albumin in BAL fluid from mice three hours after LPS (125 μg/kg i.n.) challenge (g) or S. pneumoniae challenge (h) with and without the PAR₁ antagonist RWJ-58259 (5 mg/kg) dosed therapeutically i.p. after 30 min.

FIG. 17. Healthy human volunteers were challenged with nebulized 0.9% saline or in sterile saline (final LPS dose was 50 μg) and BAL fluid collected 6 hours later. CCL7 protein levels in BAL fluid from saline-treated or LPS-challenged human volunteers were measured by ELISA (a). Human neutrophil chemotaxis was measured across 5 μm membranes (ChemoTX, NeuroProbe) in response to recombinant human (rh) CXCL8 or rhCCL7 (b). Neutrophil chemotaxis towards LPS-treated human BAL fluid was measured in the presence of neutralizing anti-CXCL8 or anti-CCL7 antibodies (c). BAL fluid was collected from patients suffering with ALI on intensive care units. CCL7 protein (d) and CCL2 protein (f) in ALI BAL fluid was measured by ELISA. Neutrophil chemotaxis towards BAL fluid obtained from patients with ALI was measured in the presence of neutralizing anti-CXCL8 and ant-CCL7 antibodies (e). Statistical analysis was performed using ANOVA ** (a) and paired Student's t-test (b,c,e,f).

FIG. 18. CC-chemokine receptor expression of neutrophils isolated from the blood and lung. Mice were administered with LPS (125 μg/kg i.n.) or without (naïve) and blood and lungs isolated and single cell suspensions prepared. Cells were stained for Ly6G and the neutrophil population specifically gated (Ly6Ghigh against FSc). The expression of CCR1, CCR2, CCR3 and CXCR2 on neutrophils was calculated and represented as dot plots. Neutrophils isolated from the blood (A), naïve lungs (B) and LPS-treated lungs (C) were analysed and the percentage of chemokine receptor positive cells calculated (D). Data were analysed by one way ANOVA with Newman-Keuls post hoc test: *p<0.05.

### DETAILED DESCRIPTION OF THE INVENTION

### Blocking CCL7

CCL7 antagonists of the invention block the function of CCL7. Blocking of CCL7 encompasses any reduction in its activity or function that results in an effect advantageous for the treatment and/or prevention of ALI/ARDS.

Typically, the blocking of CCL7 results in a reduction in neutrophilia, a reduction in neutrophil infiltration, a reduction in neutrophil accumulation and/or a reduction in the total number of neutrophils within the lung, particularly within the alveolar spaces. Preferably this reduction is mediated by the blocking of CCL7 reducing neutrophil migration or neutrophil chemotaxis. The migration of neutrophils may be measured by assays which quantify the number of Ly6G+ neutrophils. Blocking CCL7 may also decrease the ability of neutrophils to respond to classical chemoattractants such as CXCL8.

Blocking encompasses both total and partial reduction of CCL7 activity or function, for example total or partial prevention of the CCL7/CCR1, CCL7/CCR2, CCL7/CCR3, interactions. For example, a blocking antagonist of the invention may reduce the activity of CCL7 by from 10 to 50%, at least 50% or at least 70%, 80%, 90%, 95% or 99%.

Blocking of CCL7 activity or function can be measured by any suitable means. For example, blocking of the CCL7/CCR1, CCL7/CCR2, CCL7/CCR3, interaction can be determined by measuring the effect on Phosphorylation of the CCRs, phosphorylation of their associated G-coupled proteins or phosphorylation of ERK1 or ERK2. CCR activation can also be measured by Ca²⁺ mobilization. Neutrophil activation can also be measured by, for example, measuring myeloperoxidase (MPO) activity as a measure of neutrophil activation, elastase or matrix metalloproteinase (MMP, e.g. any of MMP 1-9) release as a measure of neutrophil activation, shape change assay or release of reactive oxygen species (ROS) as a measure of neutrophil activation.

Blocking of CCL7 can also be measured via assays that measure migration or chemotaxis, for example neutrophil chemotaxis assays such as Bowden chamber assays or ChemoTX assays.

Blocking of CCL7 can also be measured via assays that measure the effect of CCL7 on the alveolar-capiliary barrier, such as assays measuring the level of serum albumin in broncoalveolar lavage (BAL) fluid. Preferably blocking CCL7 reduces disruption of the alveolar-capilary barrier and so decreases the level of serum albumin in BAL fluid

Blocking may take place via any suitable mechanism, depending for example on the nature (see below) of the antagonist used, e.g. steric interference in any direct or indirect CCL7/CCR1, CCL7/CCR2, CCL7/CCR3, interaction or knockdown of CCL7 expression.

Blocking PAR₁ and/or Other Members of the PAR1-CCL7 Axis

PAR₁ and/or other members of the PAR₁-CCL7 axis can also be blocked in the manner described above in relation to CCL7. Suitable PAR₁-CCL7 axis member targets include CCR1, CCR2 and CCR3.

Blocking of PAR₁ can also be measured via assays that measure the presence or level of particular cytokines or chemokines, preferably. Typically blocking of PAR₁ reduces the expression of CCL7 (protein or mRNA), but has no effect on CXCL1 expression. Blocking of PAR₁ may also be measured by a reduction in CXCL10 and/or CXC3CL1 expression, even though blocking either of CXCL10 or CXC3CL1 does not affect neutrophil migration.

Blocking of PAR₁ may also be measured by assays that measure the number of macrophages. Typically, blocking of PAR₁ decreases the influx of macrophages to a tissue.

Blocking of PAR₁ may also be measured by assays that measure the presence or level of thrombin-anti-thrombin (TAT).

### Blocking CCL2

CCL2 can also be blocked in the manner described above in relation to CCL7. Blocking CCL2 encompasses any reduction in its activity or function that results in an effect advantageous for the treatment and/or prevention of ALI/ARDS.

Typically, the blocking of CCL2 results in a reduction in neutrophilia, a reduction in neutrophil infiltration, a reduction in neutrophil accumulation and/or a reduction in the total number of neutrophils within the lung, particularly within the alveolar spaces. Preferably this reduction is mediated by the blocking of CCL2 reducing neutrophil migration or neutrophil chemotaxis.

Measurement of CCL2 blocking may be achieved using any of the techniques described herein in relation to CCL7 antagonism. Any suitable CCL2 antagonist may be used. A CCL2 antagonist may be of any type described herein. For example, a CCL2 antagonist of the invention may be selected from peptides and peptidomimetics; antibodies; small molecule inhibitors; double-stranded RNA; antisense RNA; aptamers; and ribozymes. Preferred antagonists included antibodies.

### Antagonists

Any suitable antagonist may be used according to the invention, for example peptides and peptidomimetics; antibodies; small molecule inhibitors; double-stranded RNA; antisense RNA; aptamers; and ribozymes. Preferred antagonists include peptide fragments of CCL7, other PAR₁-CCL7 axis member targets such as PAR₁, CCR1, CCR2 and CCR3 and/or CCL2; antisense RNA, aptamers and antibodies.

### Peptides

Peptide antagonists of CCL7 will typically be fragments of CCL7 that compete with full-length CCL7 for binding to CCR1, CCR2 and/or CCR3 and hence antagonise CCL7. Similarly, peptide antagonists of CCL2 will typically be fragments of CCL2 that compete with full-length CCL2 for binding to its receptors, including CCR1, CCR2 and/or CCR3 and hence antagonise CCL2. Such peptides may be linear or cyclic. Peptide antagonists will typically be from 5 to 50, preferably 10-40, 10-30 or 15-25 amino acids in length and will generally be identical to contiguous sequences from within CCL7 or CCL2 but may have less than 100% identity, for example 95% or more, 90% or more or 80% or more, as long as they retain CCL7-blocking or CCL2-blocking properties. Blocking peptides can be identified in any suitable manner, for example, by systematic screening of contiguous or overlapping peptides spanning part or all of the CCL7 or CCL2 sequence. Peptidomimetics may also be designed to mimic such blocking peptides. Blocking peptides and peptidomimetics for PAR₁ and other PAR₁-CCL7 axis member targets can also be designed in the same way.

### Double-Stranded RNA

Using known techniques and based on a knowledge of the sequence of CCL7, another PAR₁-CCL7 axis member target or CCL2, double-stranded RNA (dsRNA) molecules can be designed to antagonise the target by sequence homology-based targeting of its RNA. Such dsRNAs will typically be small interfering RNAs (siRNAs), usually in a stem-loop (“hairpin”) configuration, or micro-RNAs (miRNAs). The sequence of such dsRNAs will comprise a portion that corresponds with that of a portion of the mRNA encoding the target. This portion will usually be 100% complementary to the target portion within the target mRNA but lower levels of complementarity (e.g. 90% or more or 95% or more) may also be used.

### Antisense RNA

Using known techniques and based on a knowledge of the sequence of the target, single-stranded antisense RNA molecules can be designed to antagonise targets by sequence homology-based targeting of their RNA. The sequence of such antisense will comprise a portion that corresponds with that of a portion of the mRNA encoding the target. This portion will usually be 100% complementary to the target portion within the target mRNA but lower levels of complementarity (e.g. 90% or more or 95% or more) may also be used.

### Aptamers

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Non-modified aptamers are cleared rapidly from the bloodstream, with a half-life of minutes to hours, mainly due to nuclease degradation and clearance from the body by the kidneys. Such non-modified aptamers have utility in, for example, the treatment of transient conditions such as in stimulating blood clotting. Alternatively, aptamers may be modified to improve their half life. Several such modifications are available, such as the addition of 2′-fluorine-substituted pyrimidines or polyethylene glycol (PEG) linkages.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. No. 5,654,151, U.S. Pat. No. 5,503,978, U.S. Pat. No. 5,567,588 and WO 96/38579.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Antibodies

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. Each light chain is comprised of a light chain variable region (abbreviated herein as V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR).

The constant regions of the antibodies may mediate the binding of the immunoglobulin to host tissues or factors, including various cells of the immune system (e.g., effector cells) and the first component (Clq) of the classical complement system.

An antibody of the invention may be a monoclonal antibody or a polyclonal antibody, and will preferably be a monoclonal antibody. An antibody of the invention may be a chimeric antibody, a CDR-grafted antibody, a nanobody, a human or humanised antibody or an antigen binding portion of any thereof. For the production of both monoclonal and polyclonal antibodies, the experimental animal is typically a non-human mammal such as a goat, rabbit, rat or mouse but may also be raised in other species such as camelids.

Polyclonal antibodies may be produced by routine methods such as immunisation of a suitable animal, with the antigen of interest. Blood may be subsequently removed from the animal and the IgG fraction purified.

Monoclonal antibodies (mAbs) of the invention can be produced by a variety of techniques, including conventional monoclonal antibody methodology e.g., the standard somatic cell hybridization technique of Kohler and Milstein. The preferred animal system for preparing hybridomas is the murine system. Hybridoma production in the mouse is a very well-established procedure and can be achieved using techniques well known in the art.

An antibody according to the invention may be produced by a method comprising: immunizing a non-human mammal with an immunogen comprising full-length CCL7, another PAR₁-CCL7 axis member target or CCL2, a peptide fragment of CCL7, another PAR₁-CCL7 axis member target or CCL2 or an epitope within CCL7, another PAR₁-CCL7 axis member target or CCL2; obtaining an antibody preparation from said mammal; and deriving therefrom monoclonal antibodies that specifically recognise said epitope.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody of the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

Screening methods as described herein may be used to identify suitable antibodies that are capable of binding to CCL7, another PAR₁-CCL7 axis member target, or CCL2. Thus, the screening methods described herein may be carried out using an antibody of interest as the test compound.

Antibodies of the invention can be tested for binding to CCL7, another PAR₁-CCL7 axis member target or CCL2 by, for example, standard ELISA or Western blotting. An ELISA assay can also be used to screen for hybridomas that show positive reactivity with the target protein. The binding specificity of an antibody may also be determined by monitoring binding of the antibody to cells expressing the target protein, for example by flow cytometry. Thus, a screening method of the invention may comprise the step of identifying an antibody that is capable of binding CCL7 or another PAR₁-CCL7 axis member target by carrying out an ELISA or Western blot or by flow cytometry. Antibodies having the required binding properties may then be further tested to determine their effects on the activity of CCL7, another PAR₁-CCL7 axis member target, or CCL2 as described further above.

Anti-CCL7 antibodies of the invention will have CCL7 antagonist (blocking) properties as discussed above. In one embodiment, a monoclonal antibody specifically recognises an epitope within CCL7 and blocks the activity of CCL7. In one embodiment, the monoclonal antibody specifically recognises an epitope within CCL7 and blocks the interaction between CCR1, CCR2 and/or CCR3 and CC17.

Anti-CCL2 antibodies of the invention will have CCL2 antagonist (blocking) properties as discussed above. In one embodiment, a monoclonal antibody specifically recognises an epitope within CCL2 and blocks the activity of CCL2. In one embodiment, the monoclonal antibody specifically recognises an epitope within CCL2 and blocks the interaction between CCR1, CCR2 and/or CCR3 and CC17.

Antibodies of the invention specifically recognise CCL7, another PAR₁-CCL7 axis member target or CCL2, i.e. epitopes within CCL7 or another PAR₁-CCL7 axis member target or CCL2. An antibody, or other compound, “specifically binds” or “specifically recognises” a protein when it binds with preferential or high affinity to the protein for which it is specific but does not substantially bind, or binds with low affinity, to other proteins. The specificity of an antibody of the invention for target protein may be further studied by determining whether or not the antibody binds to other related proteins as discussed above or whether it discriminates between them. For example, an anti-CCL7 antibody of the invention may bind to human CCL7 but not to mouse or other mammalian CCL7.

Antibodies of the invention will desirably bind to CCL7, another PAR₁-CCL7 axis member target or CCL2 with high affinity, preferably in the picomolar range, e.g. with an affinity constant (K_{D}) of 10 nM or less, 1 nM or less, 500 pM or less or 100 pM or less, measured by surface plasmon resonance or any other suitable technique.

Once a suitable antibody has been identified and selected, the amino acid sequence of the antibody may be identified by methods known in the art. The genes encoding the antibody can be cloned using degenerate primers. The antibody may be recombinantly produced by routine methods.

Epitopes within CCL7, other PAR₁-CCL7 axis member targets and CCL2 can be identified by methods known in the art and discussed herein, notably by systematic screening of contiguous or overlapping peptides via a “PEPSCAN” approach or by forming antibodies to peptide fragments (see above) shown to block CCL7. Epitope-containing peptides can be used as immunogens for the generation of antibodies. Preferred epitopes to which to raise antibodies include those via which CCL7 binds to its receptor. Putative sequences for CCL receptor binding based on the receptor binding of paralagous CC-chemokines. Preferred epitopes can therefore expect to be located at the N-terminal region, in the N-loop, in the 30 s-loop, as well as adjacent to the disulfide binds and in the alpha helix region.

### Antagonists of PAR₁

Known PAR₁ antagonists that can be used according to the invention include voropaxar and atopaxar. Other known PAR₁ antagonists can also be used.

### Antagonists of CCL2

Known CCL2 antagonists that can be used according to the invention include the modified chemokine MCP-1(9-76) (JEM vol. 186 no. 1 131-137) and SR16951, which is a small molecule antagonist of CCL2 (The Journal of Immunology, 2009, 182, 50.13). Other known CCL2 antagonists include C243, which is also a small molecule antagonist and mNOX-E36 (Gut. 2012 March; 61(3):416-26). Anti-CCL2 neutralizing antibodies are also commercially available. Other known CCL2 antagonists can also be used.

The activity of CCL2 may also be blocked using CCR2 inhibitors. Known CCR2 inhibitors are listed in the table below.

TABLE 1
Summary of experimental and clinical statuses of CCR2 inhibitors (Expert
Opin Investig Drugs. 2011 Jun; 20(6): 745-56)

| Manufacturer | Drug | Description |
|---|---|---|
| Merck | Compound 55, 26, 29, | Randomized, double-bind, placebo-controlled |
| | MK-0812 | study in rheumatoid arthritis, MS |
| Johnson & Johnson | Compound 1d | Arthritis, allergic asthma |
| Plizer/Incyte | INC83344, INC8-8696, | Delayed-type hypersensitivity, autoimmune |
| | INC8-3284, [symbol] -4136309 | encephalomysities, MS (Phase II trail), inflammatory |
| | | arthritis chronic inflammatory diseases, |
| | | atherosclerosis |
| Roche | RS-504393 | Insulin resistance, metabolic syncrome, fibrotic |
| | | kidney disease, diabetic, nephropsthy, hepatic |
| | | steatosis |
| BMS | BMS-741672, Compound 22 | Neuropathic pain (Phase II trail), insulin resistance |
| | | in diabetic patients, inflammatory and metabolic |
| | | diseases |
| Novartis AG | NBR-177, NBR-1282 | Rheumatoid arthritis, autoimmune diseases, |
| | | HIV infection, transplant rejection, inflammatory |
| | | diseases |
| ChemoCentryx | CCR2, CCR9, CCX-140 | Vascular restenosis, MS, metabolic diseases |
| | | (Phase I trail) |
| Glasko group | GSK-13443868, Compound 8 | Atherosclerosis |
| Tejin | Compound 11, 71. TAK-779, | Insulin resistance, metabolic diseases |
| | TEI-K03134 | |
| Millernium | MLN-1202 | MS, atherosclerosis |
| Sanwa | Propagermarium | Autheroslerosis, insulin resitance, hepatic |
| | | steatosis, diabetic nephropathy, renal florosis, |
| | | metatobolic diseases, tumor, chronic hepatitis B |

[symbol]

indicates data missing or illegible when filed

### Therapeutic Indications

The antagonists of the invention may be used to treat and/or prevent acute inflammation associated with the accumulation of neutrophils in the respiratory tract, especially in the conditions known as acute lung injury (ALI) and acute respiratory distress syndrome (ARDS).

ALI is an acute disease that affects the lungs but not necessarily the airways. ALI is characterised by a disruption in the alveolar epithelium and the capillary endothelium, collectively termed the capillary-alveolar barrier. The two main hallmarks of ALI are the accumulation of fluid and the migration of neutrophils in the alveolar airspaces. ALI is associated with a rapid disease onset involving the release of pro-inflammatory cytokines such as IL-1β and TNF and components of the coagulation system such as thrombin. ALI is thought to involve the activation of innate immune components, rather than adaptive. ARDS is a more severe form of ALI.

In a clinical setting, ALI/ARDS are characterised by hypoxemia, pulmonary oedema and radiological abnormalities, which have a rapid onset following a known clinical insult, or following new/worsening respiratory symptoms. The latest recommended definition of ALI/ARDS is as follows: a hypoxemia measure of PaO₂/FiO₂ 201-300 (mild), ≦200 (moderate), ≦100 (severe); with respiratory failure not explained by cardiac failure or fluid overload; with radiological abnormalities; and with additional physiological derangements in the severe form. This definition was proposed at the 2012 ESICM Annual Conference with input from the American Thoracic Society. However, the 1996 consensus definition is probably still the most widely used and is as follows: pulmonary wedge pressure less than 18 mmHg; with no clinical evidence of cardiac failure; and with a hypoxemia measure of PaO₂/FiO₂<300 ALI and <200 in ARDS. Either of these may be used to define ALI/ARDS for the purposes of the present invention.

Preferably, the antagonists of the present invention reduce excessive neutrophil accumulation without completely abolishing immune function. More preferably, inhibiting CCL7, another PAR₁-CCL7 axis member target or CCL2 reduces bystander tissue damage resulting from excessive neutrophilia, while at the same time retaining sufficient immunity for host defense and maintaining endothelial-epithelial barrier integrity, thus achieving a balance between reducing unwanted tissue damage and maintaining a protective immune response.

The invention therefore relates to treatment and/or prevention of acute inflammation associated with the accumulation of neutrophils in the respiratory tract. Such acute inflammation may be found in the lung airspaces, bronchi, bronchial wall or interstitial space.

The invention also relates to treatment and/or prevention of ALI/ARDS arising from any cause. These include both indirect and direct causes. Indirect causes include sepsis (septicaemia, endotoxemia), pancreatitis and tissue trauma distal to the lung. Direct causes include trauma to the lung, bacterial infection (community acquired pneumonia is the most common, of which Streptococcus pneumoniae is the most common aetiological agent, although ALI/ARDS may result from infection with other bacteria, e.g. Haemophilus influenza or Chlamydophila pneumoniae), viral infection (the most common aetiological agents being influenzavirus, coronaviruses, e.g. severe acute respiratory syndrome coronavirus (SARS-CoV) and cytomegaloviruses, which are a particular problem in the immunocompromised), and other respiratory diseases such as infant respiratory distress syndrome (IRDS), bronchiectasis (including its underlying causes, e.g. infection with Staphylococcus sp., Klebsiella sp. and Bordetella pertussis), chronic obstructive pulmonary disease (COPD) with particular relevance to acute exacerbations.

### Pharmaceutical Compositions, Dosages and Dosage Regimes

Antagonists of the invention will typically be formulated into pharmaceutical compositions, together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. the carrier is suitable for parenteral, e.g. intravenous, intramuscular, subcutaneous, intraocular or intravitreal administration (e.g., by injection or infusion). Preferably the carrier is suitable for intranasal or inhalational administration. Depending on the route of administration, the modulator may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be preferable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional active ingredients as discussed herein.

Also within the scope of the present invention are kits comprising antagonists of the invention and instructions for use. The kit may further contain one or more additional reagents, such as an additional therapeutic or prophylactic agent as discussed above.

The antagonists and compositions of the present invention may be administered for prophylactic and/or therapeutic treatments.

In therapeutic applications, modulators or compositions are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, formulations are administered to a subject at risk of a disorder or condition as described above, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject.

A subject for administration of the antagonists of the invention may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is preferred.

An antagonist of the present invention may be administered via one or more routes of administration using one or more of a variety of methods known in the art. As will be appreciated by the skilled artisan, the route and/or mode of administration will vary depending upon the desired results. Routes of administration for modulators of the invention include intravenous, intramuscular, intradermal, intraocular, intraperitoneal, subcutaneous, spinal or other parenteral routes of administration, for example by injection or infusion. The phrase “parenteral administration” as used herein means modes of administration other than enteral and topical administration, usually by injection. Alternatively, an antagonist of the invention can be administered via a non-parenteral route, such as a topical, epidermal or mucosal route of administration. Preferably, the antagonist of the invention is administered by an intranasal or inhalational route.

A suitable dosage of a modulator of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

As mentioned above, modulators of the invention may be co-administered with one or other more other therapeutic agents. For example, the other agent may be an analgesic, anaesthetic, immunosuppressant or anti-inflammatory agent.

Combined administration of two or more agents may be achieved in a number of different ways. Both may be administered together in a single composition, or they may be administered in separate compositions as part of a combined therapy. For example, the one may be administered before, after or concurrently with the other.

### Combination Therapies

As noted above, antagonists of the invention may be administered in combination with any other suitable active compound.

In particular, antagonists of different members of the PAR₁-CCL7 axis may be administered in combination, for example an antagonist of CCL7 can be administered in combination with an antagonist of PAR₁ and/or CCR1, and/or CCR2 and/or CCR3. Similarly, an antagonist of PAR₁ can be administered in combination with an antagonist of CCL7 and/or CCR1, and/or CCR2 and/or CCR3. An antagonist of CCR1 can be administered in combination with an antagonist of CCL7 and/or PAR₁, and/or CCR2 and/or CCR3. An antagonist of CCR2 can be administered in combination with an antagonist of CCL7 and/or PAR₁, and/or CCR1 and/or CCR3. An antagonist of CCR3 can be administered in combination with an antagonist of CCL7 and/or PAR₁, and/or CCR2 and/or CCR1.

The CCL7 antagonists, PAR₁ antagonists and/or other antagonists of the PAR₁-CCL7 axis may be used in combination with the CCL2 antagonists of the invention. For example an antagonist of CCL2 can be administered in combination with an antagonist of CCL7, PAR₁ and/or CCR1, and/or CCR2 and/or CCR3.

The antagonist of the invention may also be administered in combination with an antagonist of the pro-inflammatory chemokine CXCL8 (Interleukin-8; IL-8). The antagonist of the invention may also be administered in combination with an antagonist for the IL-8 receptor, CXCR1 (also known as interleukin 8 receptor alpha, IL8RA, CD181). CXCL8 is a known chemoattractant for neutrophil extravasation across endothelial and epithelial surfaces. (Grommes, J. & Soehnlein, O. Mol. Med. 17, 293-307 (2011)). The CXCL8 or CXCR1 antagonist may be, for example selected from peptides and peptidomimetics; antibodies, preferably monoclonal antibodies; small molecule inhibitors; double-stranded RNA; antisense RNA; aptamers and ribozymes as discussed herein in relation to CCL7, other PAR₁-CCL7 axis member targets and CCL2. The effect of inhibition of CCL7 and CXCL8 and/or CXCR1, for example on neutrophil migration or chemotaxis, by use of a combination of a CCL7 and a CXCL8 and/or CXCR1 antagonist may be additive or synergistic compared to the effect of CCL7 and CXCL8 and/or CXCR1 inhibition alone. Similarly, antagonists of CXCL8 and/or CXCR1 may be used in combination with CCL2 antagonists of the invention in the same way.

The following Examples illustrate the invention.

### Examples

### 1. PAR₁ Contributes to Acute Lung Inflammation

PAR₁ is the main receptor for the coagulation factor thrombin and is critical in orchestrating the interplay between coagulation and inflammation (Chambers, R. C. Br. J. Pharmacol. 153 Suppl 1, S367-S378 (2008)). PAR₁ activation also leads to the upregulation of several proinflammatory genes that mediate neutrophil recruitment into the lungs (Mercer, P. F. et al Ann. N. Y. Acad. Sci. 1096, 86-88 (2007)).

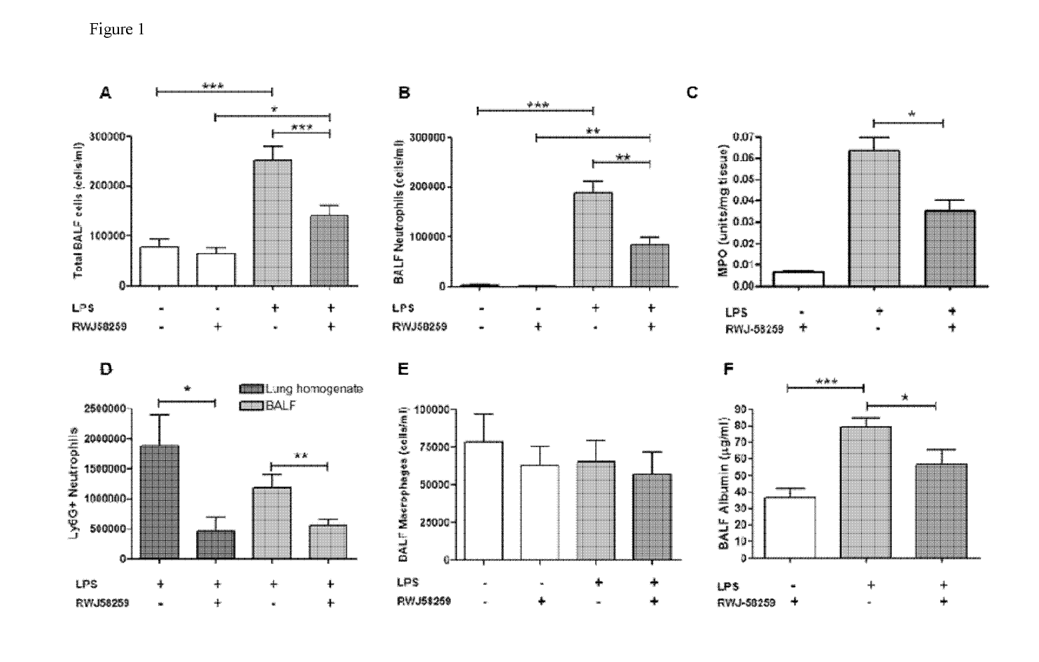

To determine the role of PAR₁ in acute lung inflammation mice were treated with a specific PAR₁ antagonist (5 mg/kg, a kind gift from Claudia Derian, Johnston and Johnson Pharmaceutical Research & Development, USA) following intranasal challenge with LPS (125 μg/kg). Experiments were conducted with local ethical approval in accordance with the Home Office, UK. Female BALB/c mice (6-8 weeks; Charles River, UK) were anaesthetized (5% isofluorane) and challenged with LPS in sterile saline (125 μg/kg, 50 μl i.n.; Escherichia coli 0127:B8; Sigma, UK). LPS caused a significant increase in total cell and neutrophil recruitment into alveolar spaces (FIG. 1a, b), events that are indicative of the early stages of acute inflammation (Summers, C. et al Trends Immunol. 31, 318-324 (2010)). Total and differential counts were quantified following cytospin.

Mice were injected i.p with the PAR₁ antagonist RWJ-58259 30 min after LPS administration. Antagonism of PAR₁ immediately after the onset of acute lung inflammation significantly decreased total cell and neutrophil numbers in the airspaces (FIG. 1b), showing that PAR₁ plays a central role in the induction of acute lung inflammation. The decrease in airspace neutrophilia was confirmed by flow cytometry, which demonstrated a decrease in Gr-1+(F4/80−) neutrophils recovered from BAL fluid and in whole lung preparations (FIG. 1d). PAR₁ antagonism reduced LPS-induced neutrophil myeloperoxidase activity as detected in lung homogenates (FIG. 1c).

Macrophage numbers in BAL fluid remained unchanged in all treatment groups, indicating that PAR₁ antagonism did not affect early macrophage recruitment into BAL fluid (FIG. 1e). In order to examine the effect of PAR₁ antagonism on LPS-induced disruption of the alveolar-capillary barrier, serum albumin levels were measured in BAL fluid recovered from saline and LPS challenged mice. Serum albumin levels were increased in the BAL fluid of LPS challenged mice, and were significantly decreased (p=0.004) following PAR₁ antagonism (FIG. 1f). These data therefore demonstrate that PAR₁ signalling influences early neutrophilic inflammation and alveolar-capillary barrier disruption in a model of acute lung inflammation.

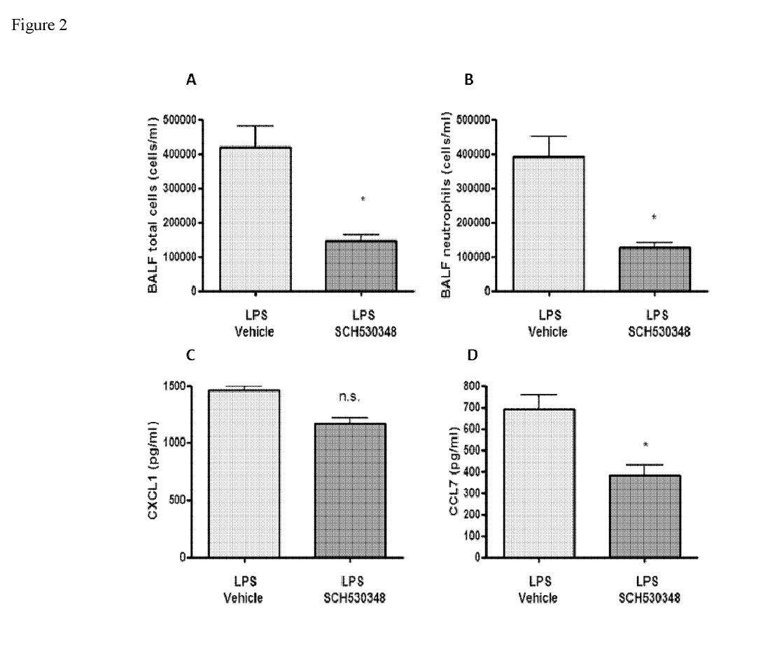

Similar results were observed when a different PAR₁, SCH530348, was used. Mice were killed three hours after LPS (125 μg/kg i.n.) challenge with and without the specific PAR₁ antagonist SCH530348 (10 mg/kg) dosed therapeutically i.p. immediately after LPS challenge. Lungs were lavaged and total cells and neutrophils counted using a haemocytometer and cytospin preparation. Data were analysed by one way ANOVA with Neuman-Keuls Post Hoc test: *p<0.05. Again, antagonism of PAR₁ immediately after the onset of acute lung inflammation significantly decreased total cell (FIG. 2A) and neutrophil (FIG. 2B) numbers in the airspaces

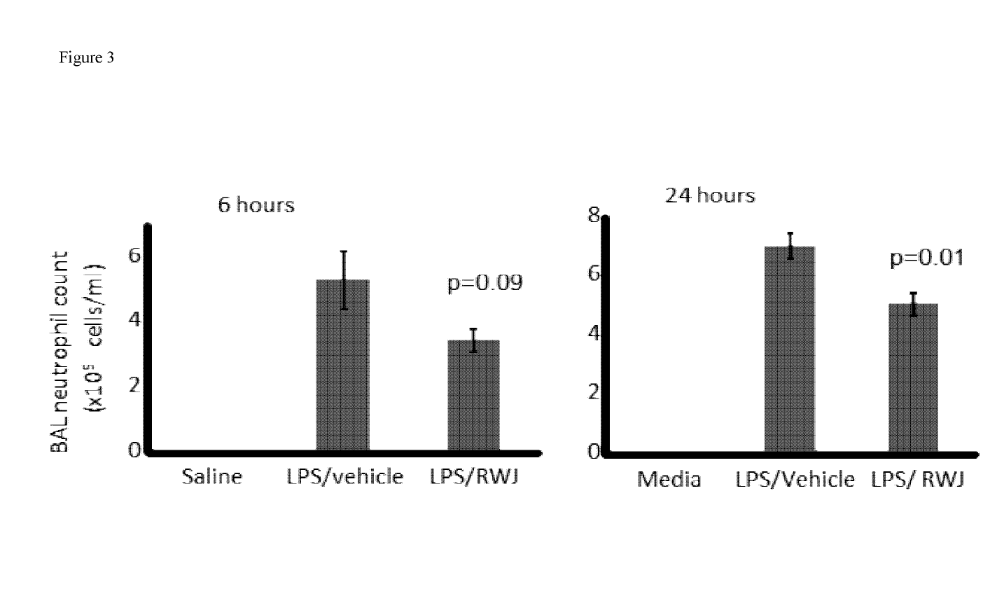

Furthermore, PAR₁ antagonism with RWJ-58259 also reduced neutrophil recruitment 6 h and 24 h following LPS challenge (FIG. 3).

In order to examine the influence of PAR₁ during host defense and in a model of bacteria-induced ALI, mice were challenged with S. pneumoniae (5×10⁶ CFU/mouse, i.n.), which is the most common infectious agent responsible for ALI. Mice were inoculated with 50 μl S. pneumoniae (serotype 19, 5×10⁶ CFU/mouse i.n.). 3 hours later, animals were sacrificed (urethane i.p. 20 g/kg), endotracheally cannulated and bronchoalveolar lavage performed (1.5 ml, PBS). Total and differential counts were quantified following cytospin and albumin levels measured by ELISA (Bethyl Laboratories Inc, USA).

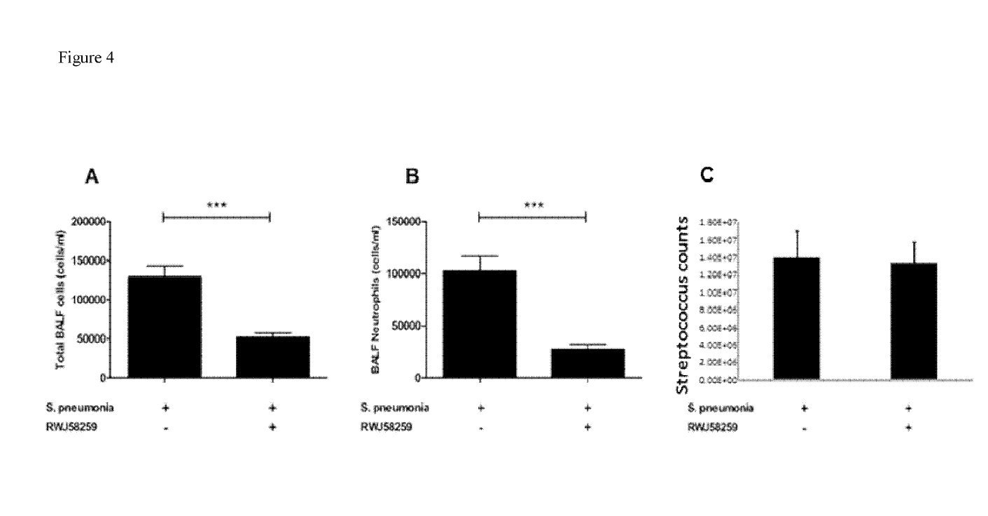

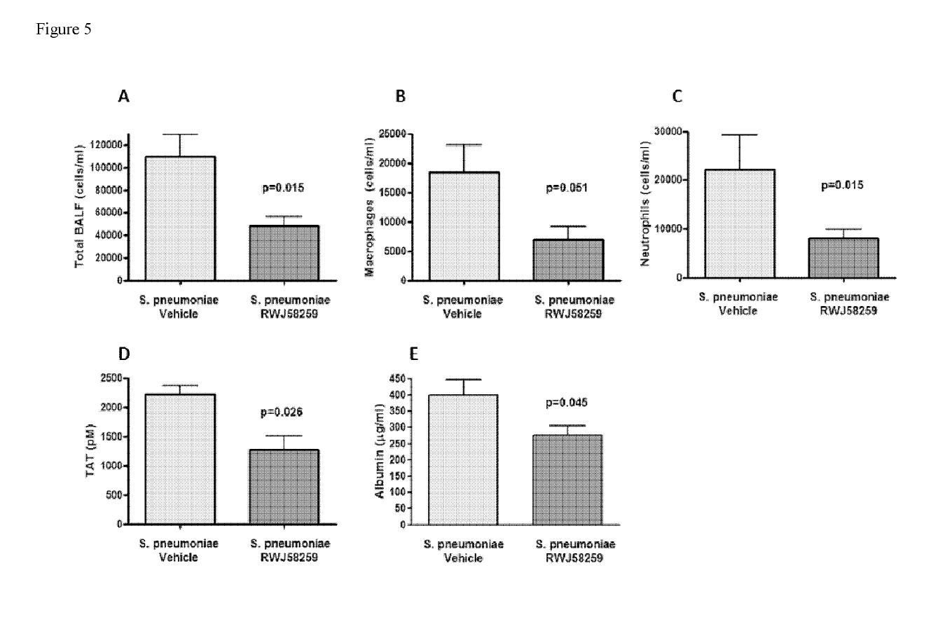

Infection with S. pneumoniae caused an increase in total leukocyte and neutrophil accumulation within airspaces, all characteristic of the early stages of pulmonary bacterial infection and bacteria-induced ALI. In mice treated with the PAR₁ antagonist, total cellular infiltration and neutrophil accumulation were reduced (FIG. 4a, b). Such a significant reduction in neutrophilia could have detrimental consequences on host defense, particularly against bacterial infections that are often concomitant during ALI. However, PAR₁ antagonism did not compromise host defense, as S. pneumoniae colony counts recovered from whole lung were unaffected by PAR₁ antagonist treatment (FIG. 4c).

Similar results were observed with a different strain of S. pneumoniae (serotype 2, clinical isolate D39, 50 μl/mouse, 5×10⁶ CFU/mouse i.n.) with and without the PAR₁ antagonist RWJ-58259 (5 mg/kg) dosed therapeutically i.p. after 30 minutes (FIG. 5a, c). PAR₁ antagonism was also found to decrease the number of BAL macrophages (FIG. 5b). Bronchoalveolar lavage fluid was collected and levels of thrombin-anti-thrombin (TAT) and serum albumin were quantified by ELISA (FIG. 4d, e). PAR₁ antagonism was found to reduce PAR₁ mediated disruption of the alveolar-capillary barrier. Panels show mean values for n=5/group from two separate experiments. Data were analysed by one way ANOVA with Neuman-Keuls Post Hoc test: ***p<0.0001, *p<0.05.

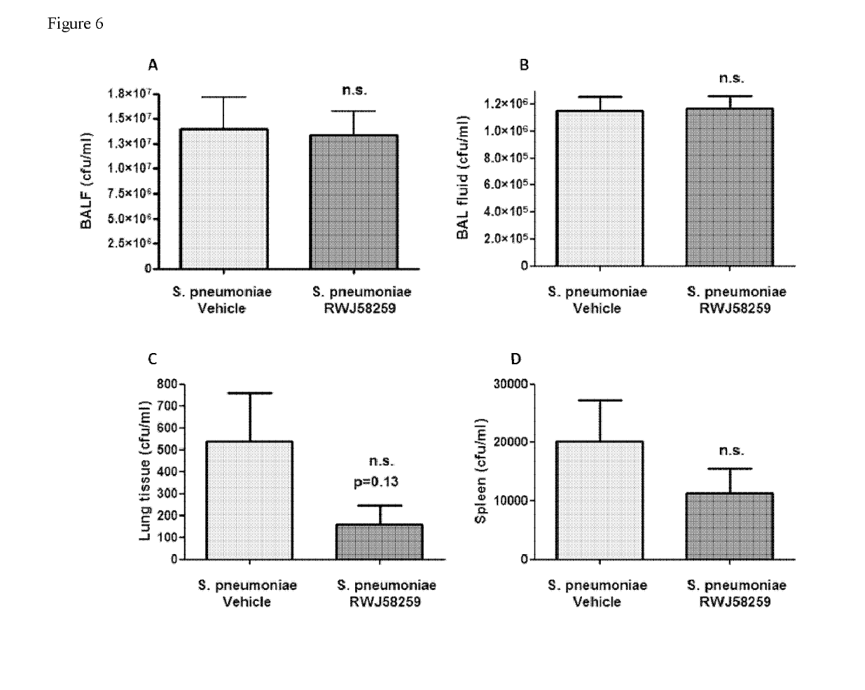

Results obtained using this second S. pneumonia strain also demonstrated that PAR₁ antagonism did not compromise host defense, as S. pneumoniae colony counts recovered from BALF obtained after three hours (FIG. 6a) and 24 hours (FIG. 6b) were unaffected by PAR₁ antagonist treatment. Bacterial invasive disease was measured by cfu in the lung (FIG. 6c) and the spleen (FIG. 6d) after 24 hours. Data were analysed by one way ANOVA with Neuman-Keuls Post Hoc test: n.s. not significant. Again, the counts were unaffected by PAR₁ antagonist treatment, indicating that PAR₁ antagonism does not adversely affect the immune response to S. pneumonia infection.

### 2. PAR₁ Signalling Mediates CCL7 Expression

To investigate the mechanism by which PAR₁ signalling influences acute lung inflammation, the effect of LPS challenge and subsequent PAR₁ antagonism on neutrophil-specific chemokine levels in lung homogenates was examined. The CXCR2 ligands, CXCL1 (keratinocyte-derived chemokine, KC) and CXCL2/CXCL3 (macrophage inflammatory protein-2, MIP-2α/β), which are functional homologues of human CXCL8 (IL-8) and CXCL2/CXCL3 (growth-related oncogenes GRO-β/GRO-γ), have been implicated as the primary mediators of neutrophil recruitment into inflamed tissue.

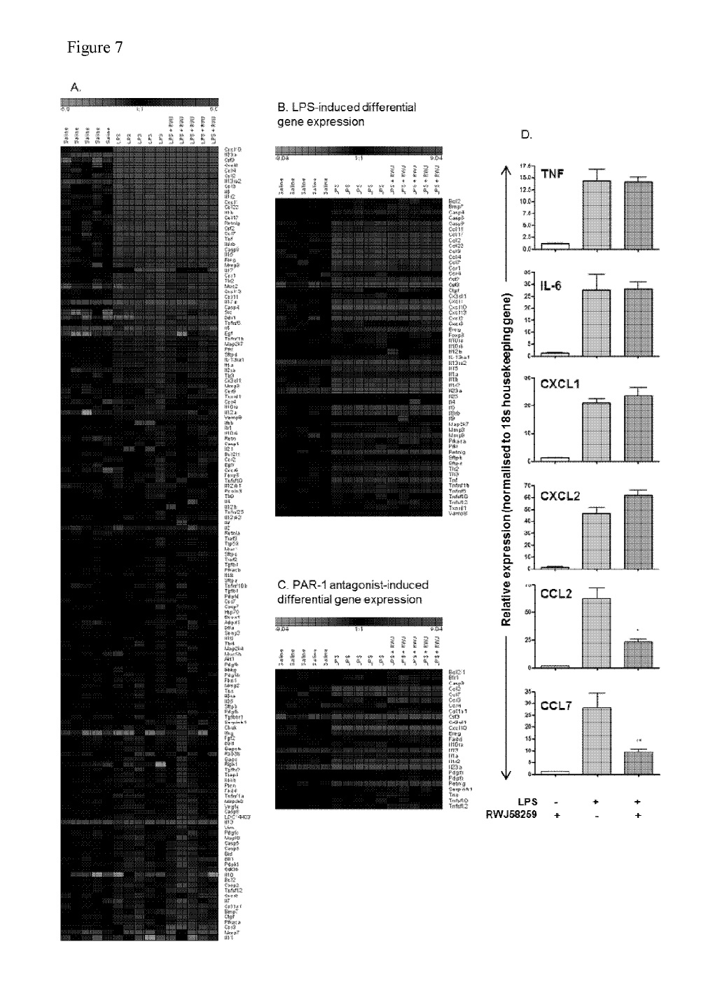

In this study, LPS challenge significantly increased the levels of these chemokines, as previously demonstrated (Huber, A. R. et al Science 254, 99-102 (1991)). However, levels of CXCL1 and CXCL2 were unaffected by PAR₁ antagonism (FIG. 7a, b), suggesting that these chemokines are not regulated by PAR₁. Similarly, PAR₁ antagonism did not attenuate the expression of the proinflammatory cytokines, TNF and IL-6 (FIG. 7c, d), which are characteristically associated with acute inflammation. Cytokine and chemokine levels were measured by ELISA.

These data led us to conclude that PAR₁ mediated acute inflammatory responses are not associated with the induction of classical neutrophil chemoattractants or proinflammatory cytokines.

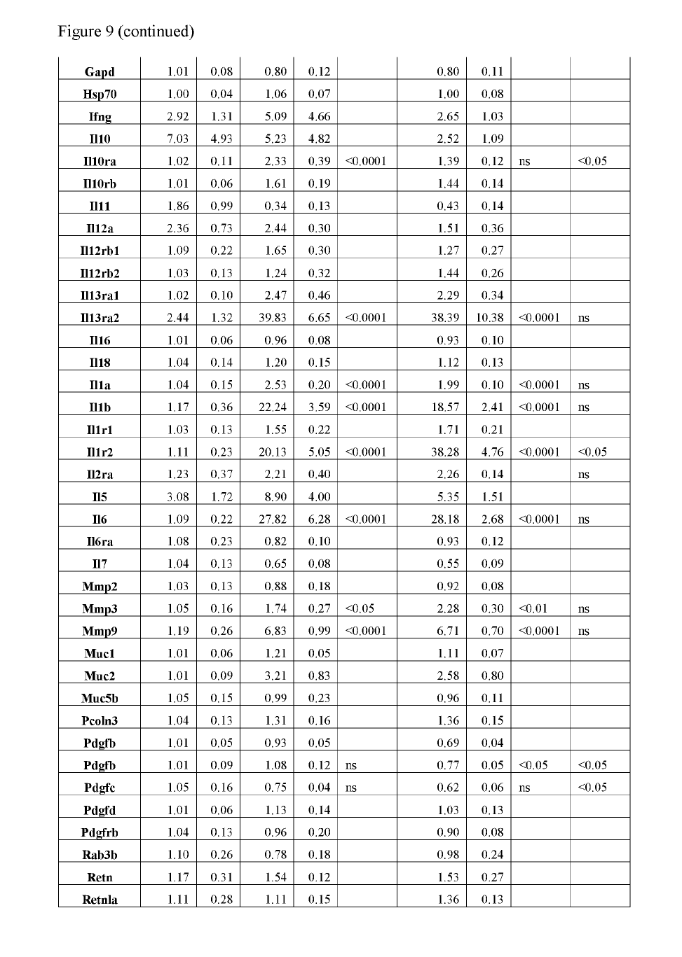

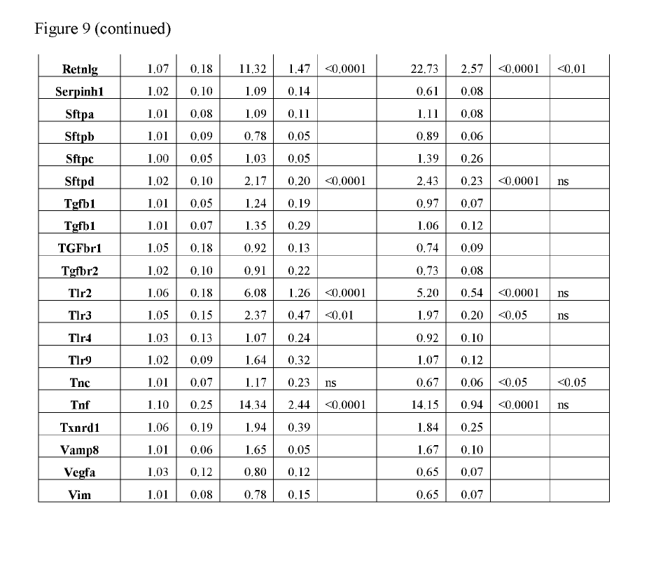

In order to identify potential PAR₁-regulated cytokine/chemokine candidates involved in LPS-induced ALI, a low density array (LDA) designed to profile 151 inflammatory mediators was used (FIG. 7a, FIG. 9). Total RNA was extracted from pulverised frozen pulverised lung using TRIzol (see manufacture's protocol (Invitrogen)), DNase treated using a DNA free kit (Ambion) and cDNA synthesised from 1 μg RNA/per sample using a Superscript kit (Invitrogen). Expression levels of known inflammatory mediators were analysed in cDNA using Taqman low density array qPCR chips and normalized to 18 s. Transcript data was analysed using the Gene Expression Similarity Suite (Genesis) software⁵¹ and data represented as a heat map following log₂ transformation and normalisation. Relative fold-difference in expression was calculated using the ΔΔT method with the saline treated group as the calibrator reference. 51 genes were found to be differentially expressed in lung tissue following challenge with LPS (FIG. 7b, FIG. 9). 32 genes were demonstrated to be significantly upregulated following LPS challenge (FIG. 7b, FIG. 9), including several chemokines. The differential gene expression profile included the upregulation of several genes known to be important for the generation of inflammatory responses, such as TNF, interleukins, CXC chemokines and CC chemokines, including CCL2, CCL3, CCL4, CCL7, CCL22, CXCL1, CXCL2, CXCL10, CXCL13 and CX3CL1. Further analysis revealed that 25 genes exhibited decreased expression following PAR₁ antagonism (FIG. 7c).

In agreement with the previous protein data, the expression of the neutrophil-specific chemokines, CXCL1 and CXCL2, and the cytokines, TNF and IL-6, increased following LPS injury but were unaffected by PAR₁ antagonism (FIG. 7d), suggesting that these cytokines/chemokines are not regulated downstream of PAR₁ signalling in this model of LPS-induced lung inflammation. Of the 32 genes found to be upregulated following challenge with LPS, PAR₁ antagonism decreased the expression of two closely related CC-chemokines CCL2 (MCP-1) and CCL7 (MCP-3) (FIG. 7d), which have not been conventionally associated with neutrophil recruitment. Instead, these chemokines are known to induce the egress of monocytes from the bone marrow and recruit monocytes/macrophages into inflamed tissue.

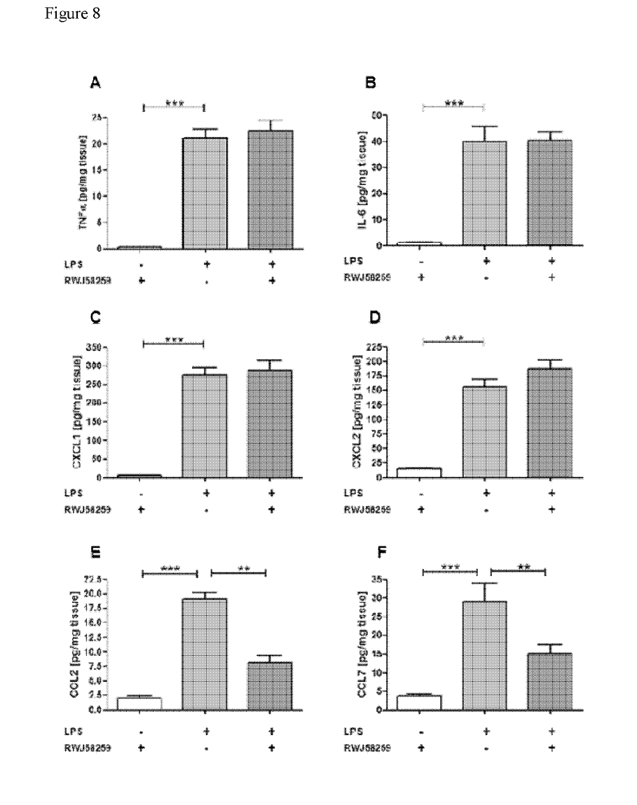

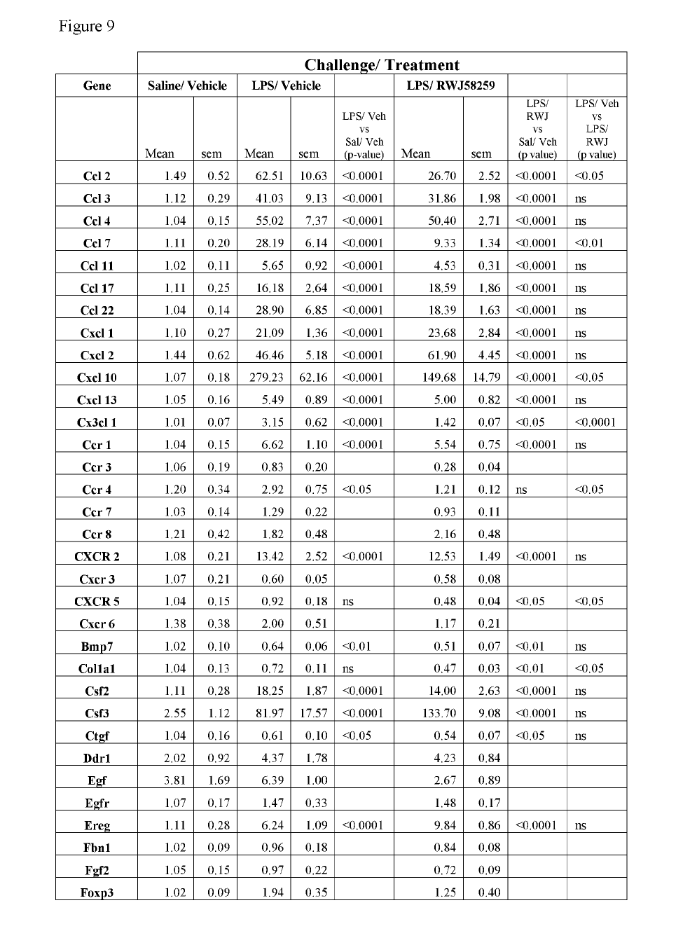

In order to confirm the LDA analysis of mRNA expression, the level of protein in lung homogenates was measured. Treatment with LPS significantly increased the expression of TNF, IL-6, CXCL1 and CXCL2 (FIG. 8a, b, c, d), consistent with observations following LDA analysis, but the expression of these proteins was not affected by PAR₁ antagonist treatment. Similarly, LPS challenge increased the expression of CCL2 and CCL7 (FIG. 8e, f) and, as observed in the LDA analysis, PAR₁ antagonist treatment also decreased the expression of these chemokines at the protein level. These data provide strong support to the notion that PAR₁ signalling plays an important role in regulating CCL2 and CCL7 expression following LPS-induced lung inflammation, and that these chemokines may directly influence neutrophil migration.

### 3. Acute Neutrophilic Inflammation is Dependent on CCL7

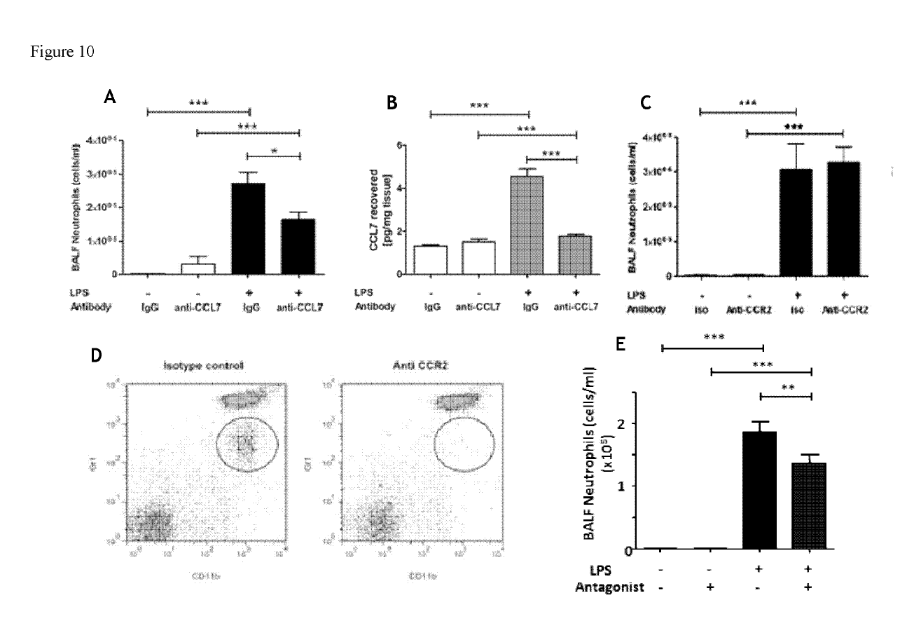

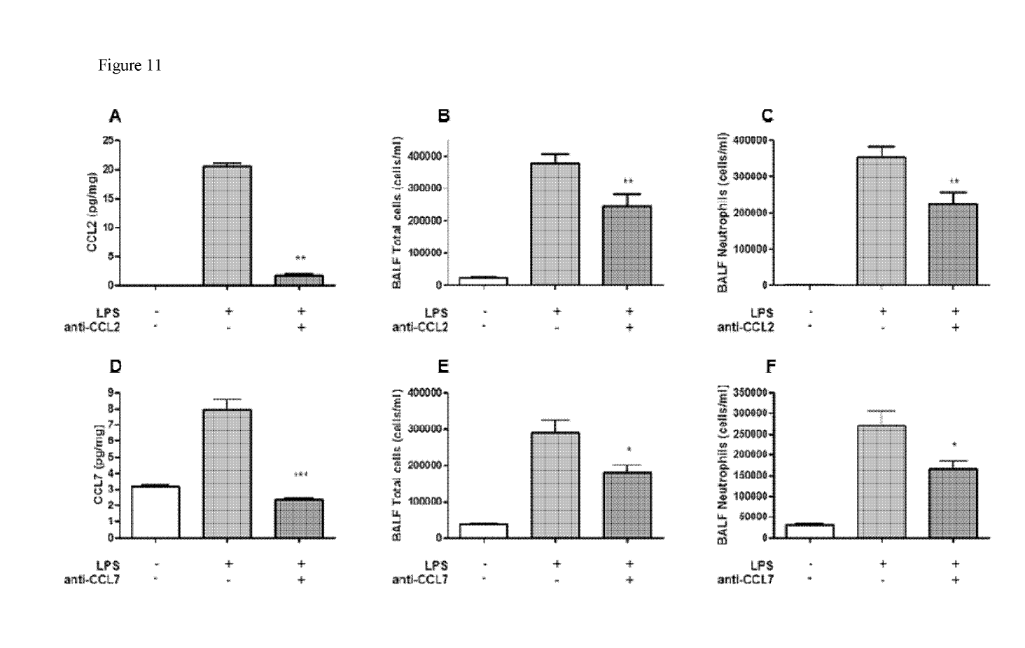

In order to examine the potential roles of CCL2 and CCL7 in LPS-induced lung inflammation, we used specific neutralizing antibodies to block these chemokines. Neutralizing antibodies were administered within the LPS challenge volume and lung homogenates analysed after 3 h. Treatment with the CCL2 or CCL7 neutralizing antibodies reduced respective chemokines to basal levels (FIG. 10B, 11A, 11D), and therefore confirmed—effective target engagement. Administration of anti-CCL2 antibody significantly decreased both the total cell number and the number of neutrophils isolated from BAL fluid following challenge with LPS (FIG. 10A, 11B, 11C) Similarly, administration of anti-CCL7 antibody also significantly reduced total cell and neutrophil accumulation into airspaces (FIG. 11E, 11F). Taken together, these data led us to conclude that both CCL2 and CCL7 influence early total leukocyte and neutrophil accumulation into the inflamed lung.

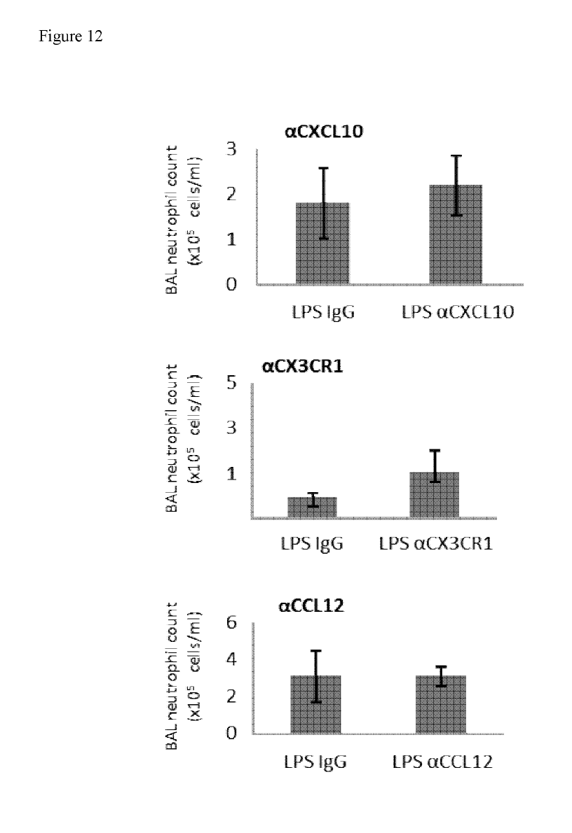

Since LDA analysis indicated that the LPS induced chemokines CXCL10 and CX3CL1 may be responsive to treatment with the PAR₁ antagonist, in vivo neutralization experiments were also performed with antibodies to CXCL10 or CX3CL1. No decrease in neutrophil accumulation was observed following neutralisation of CXCL10 or CX3CL1 (FIG. 12), suggesting that these chemokines are not directly involved in neutrophil migration into the LPS-inflamed lung.

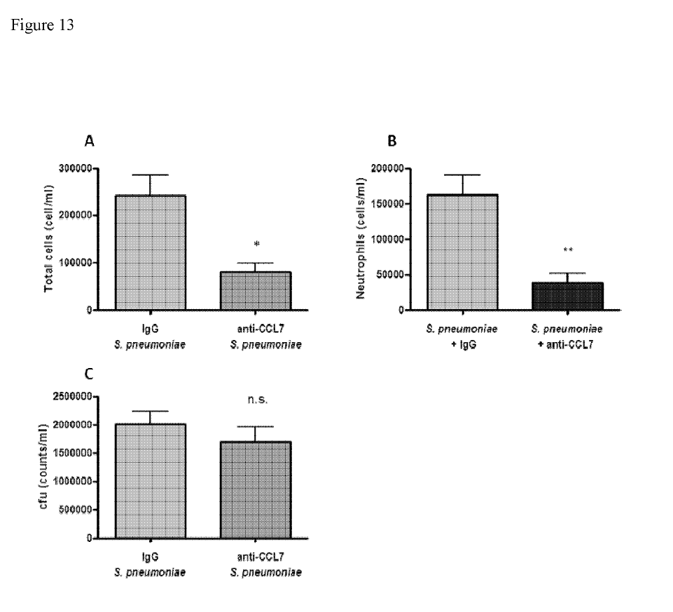

Similar results were obtained when inflammation was triggered by infection with S. pneumoniae (serotype 2, clinical isolate D39, 50 μl/mouse, 5×10⁶ CFU/mouse i.n.), rather than by LPS challenge. Mice were inoculated with S. pneumoniae with and without specific neutralizing antibody to CCL7 (10 μg/mouse i.n. within challenge volume). Lungs were lavaged (1.5 ml PBS total) and total BAL fluid leukocytes (A), and neutrophils (B) were quantified. Bacteria (cfu) recovered from the BALF were also counted (C). Data were analysed by one way ANOVA with Neuman-Keuls Post Hoc test: **p<0.001, *p<0.05. Treatment with the CCL7 neutralizing antibody significantly reduced total cell and neutrophil accumulation into airspaces (FIG. 13a, b), demonstrating that CCL7 plays an important role in the recruitment of neutrophils into the lung. Similarly to PAR₁ antagonism, the CCL7 neutralizing antibody did not negatively impact the immune response of the test mice to the S. pneumoniae infection, because the cfu counts did not differ significantly between the CCL7 neutralizing antibody treated and untreated mice.

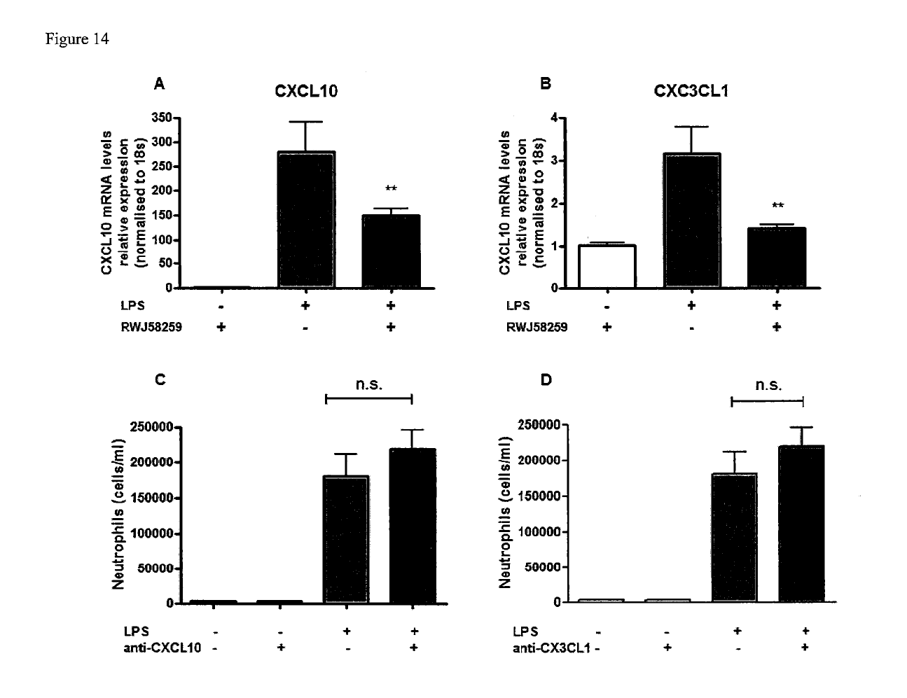

Since LDA analysis indicated that the LPS induced chemokines CXCL10 and CX3CL1 may also be responsive to treatment with the PAR₁ antagonist, in vivo neutralization experiments were also performed with antibodies to CXCL10 or CX3CR1. No decrease in neutrophil accumulation was observed following neutralisation of CXCL10, CX3CR1 or the related CC-chemokine CCL12 (FIGS. 14 and 13), strongly suggesting that these chemokines are not directly involved in neutrophil migration into inflamed lung.

In order to further exclude a role for CCR2, mice were also treated with a blocking antibody to the CCL2 receptor CCR2 (Bruhl, H. et al Arthritis Rheum. 56, 2975-2985 (2007)). Treatment of mice with this antibody had no effect on LPS-induced neutrophil accumulation (FIG. 10C), further suggesting that the CCR2 is not critical for neutrophil recruitment. Effective target engagement was confirmed by demonstrating that Gr-1+/CD11b+ monocytes in the systemic circulation, known to express CCR2 (Bruhl, H. et al Arthritis Rheum. 56, 2975-2985 (2007)), were successfully depleted (FIG. 10d). Therefore, these data demonstrate that neutrophil recruitment is not dependent on CCR2 or the presence of circulating monocytes.

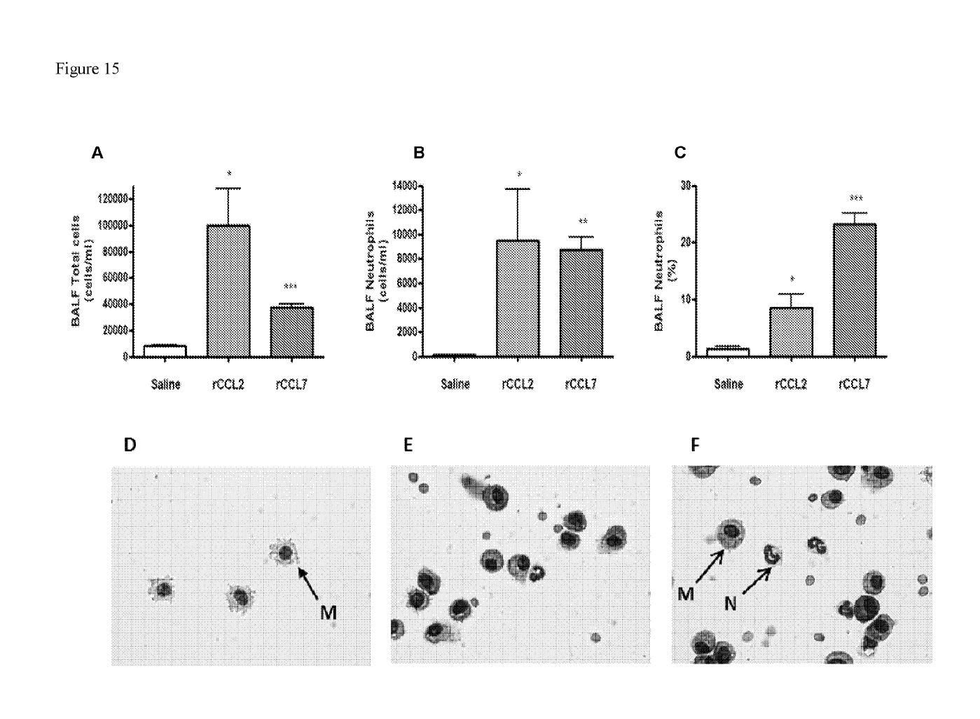

In order to determine the whether CCL2 and CCL7 are able to directly recruit leukocytes into the lung, recombinant CCL2 or CCL7 was administered into the lungs of naïve mice and sampled the BAL fluid after 3 h. Direct instillation of either rCCL2 or rCCL7 increased the total cell number recovered from BAL fluid compared to saline treated controls (FIG. 15A) The administration of rCCL2 induced the recruitment of a greater number of leukocytes compared to rCCL7 (FIG. 15A). Furthermore, administration of rCCL2 or rCCL7 also resulted in the recruitment of a neutrophils into lung airspaces (FIG. 15B) When expressed as a percentage of total cells recovered from BAL fluid, the data revealed that rCCL7 promoted a preferential accumulation of neutrophils compared to rCCL2 (FIG. 15C), although total neutrophil numbers were similar. Differential cell counts were performed on cytospin preparations following saline (FIG. 15D), rCCL2 (FIG. 15E) or rCCL7 (FIG. 15F) administration. These data reveal that both CCL2 and CCL7 are able to attract neutrophils into the lung in the absence of any underlying inflammation.

To assess whether the CC-chemokine receptor CCR1 mediates CCL7-dependent neutrophilic lung inflammation, mice were treated with an antagonist to CCR1 following LPS challenge (FIG. 10e) A reduction in airspace neutrophilia was observed following CCR1 treatment.

Taken together, these data demonstrate that CCL7 is an important chemokine for the migration of neutrophils into airspaces during acute lung inflammation and further that neutrophil migration is, at least in part, dependent on CCR1.

### 4. CCL7 Release from Bronchial Epithelial Cells

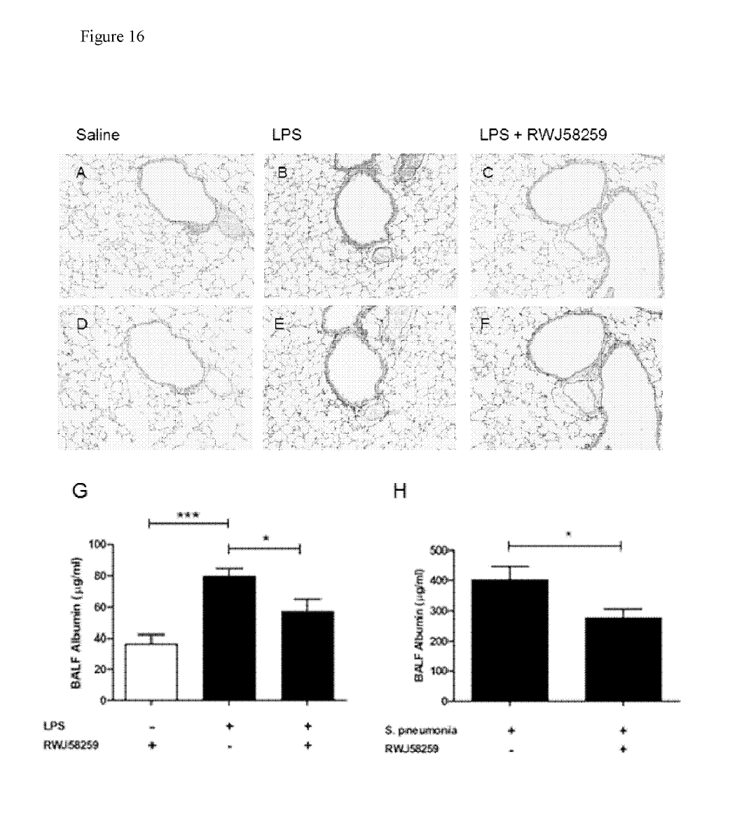

The observation that neutrophil migration downstream of PAR₁ activation was mediated by the non-classical neutrophil chemokine, CCL7, was unexpected. In order to determine the cellular source of CCL7 the immunolocalization of CCL7 and Gr-1+ neutrophils in serial lung sections from saline treated and LPS-challenged mice was examined. In saline treated control lung, there was weak CCL7 staining which was mainly restricted to the bronchial epithelium (FIG. 16a). CCL7 immunostaining was markedly increased in response to LPS injury and was also predominantly associated with the bronchial epithelium (FIG. 16b). Only weak immunolocalization was detected in the alveolar epithelium. Notably, LPS-induced CCL7 immunoreactivity was reduced in PAR₁ antagonist treated mice (FIG. 16c). Gr-1 specific immunostaining was next performed in order to detect neutrophils in serial lung sections. No Gr-1 staining could be detected in saline treated controls (FIG. 16d). Following challenge with LPS, Gr-1+ cells increased, particularly in areas exhibiting strong CCL7 immunoreactivity (FIG. 16e). As expected, PAR₁ antagonism decreased Gr-1 immunoreactivity (FIG. 160 in mouse lungs challenged with LPS.

Taken together, these studies strongly support the notion that CCL7 production and release by the bronchial epithelium following LPS injury promotes neutrophil accumulation into the inflamed lung. The attenuation of CCL7 and Gr-1 positivity in PAR₁ antagonist treated mice is also consistent with PAR₁-CCL7 axis playing an important role in the regulation of neutrophil recruitment into inflamed lungs.

In order to examine the effect of PAR₁ antagonism on LPS-induced disruption of the epithelial-endothelial barrier, serum albumin was measured in the BAL fluid recovered from LPS challenged mice. Serum albumin is normally only detected in the pulmonary vasculature and the systemic circulation and not in healthy airspaces. LPS induced an increase in serum albumin in the BAL fluid of challenged mice, indicative of barrier disruption. However, PAR₁ antagonism significantly reduced (p=***) BAL fluid serum albumin and therefore decreased epithelial-endothelial barrier disruption (FIG. 16g).

In order to further assess the influence that PAR₁ has on epithelia/endothelial barrier integrity, serum albumin levels were measured in BAL fluid following S. pneumoniae challenge (FIG. 16h). Treatment with a PAR₁ antagonist decreased BAL fluid serum albumin following challenge with S. pneumoniae, demonstrating that PAR₁ is an important mediator of bacterial-induced alveolar-capillary barrier disruption.

Taken together, these data provide compelling evidence that PAR₁ signalling contributes to the inflammatory response following direct lung injury and that antagonism of this receptor reduces excessive inflammation and tissue damage without compromising host defense.

### 5. CCL7 Regulates the Chemotaxis of Human Neutrophils During ALI

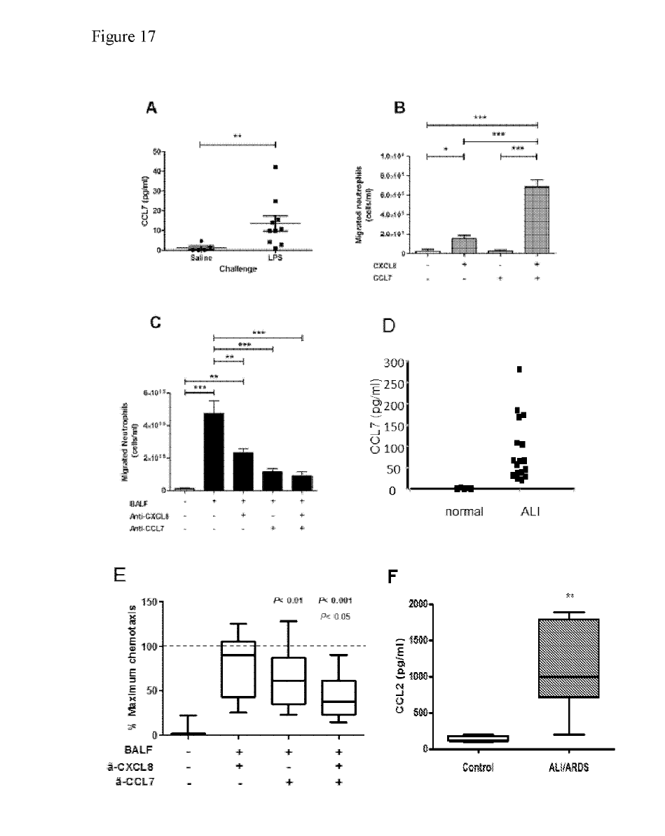

Although it is widely accepted that CXCL8 (IL-8) is an important chemokine in human neutrophilic lung disease (Miller, E. J. et al Am. Rev. Respir. Dis. 146, 427-432 (1992)), it is far from clear whether other chemokines are associated with disease pathogenesis. In order to examine the potential significance of these findings to human disease, it was next examined whether CCL7 is increased in a human model of LPS-induced ALI. For these studies, CCL7 was measured by ELISA in BAL fluid from healthy volunteers challenged with LPS (50 μg) at 6 hours as previously described (Shyamsundar, M. et al Am. J. Respir. Crit Care Med. 179, 1107-1114 (2009)). It was found that CCL7 levels were significantly increased (p=0.01) in the lungs of LPS-challenged individuals compared with volunteers given control saline (FIG. 17a). Although CCL7 is not thought to be a direct neutrophil chemoattractant (Gouwy, M. et al J. Leukoc. Biol. 76, 185-194 (2004)), the possibility that CCL7 facilitates human neutrophil migration in response to classical chemoattractants was examined. To this end, extensive chemotaxis experiments were performed using freshly isolated neutrophils from the peripheral blood of human volunteers. Human neutrophils were isolated from the blood of healthy volunteers (written consent obtained under the Human Tissue Act, UK). Neutrophils were purified over a dual Histopaque gradient (Histopaque 1119, Histopaque 1088, Sigma). Cell count and purity were assessed by microscopy. ChemoTX plates (Neuro Probe) were used throughout (3 μm pores in a 96-well plate) employing 5×104 neutrophils per well. Recombinant human CXCL8 (IL-8) and CCL7 (Peprotech) were used at 50 ng/ml. Neutrophils were incubated at 37° C. in 5% CO2 and migrated cells in the lower chamber were counted after 45 min using a haemocytometer. As expected, these experiments revealed that CXCL8 (50 ng/ml) increased the migration of neutrophils (p=0.024) compared to media alone. In contrast, CCL7 (50 ng/ml) alone did not influence neutrophil migration (FIG. 17b). However, neutrophil migration was markedly increased in response to both CXCL8 and CCL7 (p=0.001) compared with CXCL8 alone, suggesting that these two chemokines synergistically enhance neutrophil chemotaxis.

The contribution of these chemokines to the neutrophil chemotactic activity of the BAL fluid taken from LPS-treated volunteers using chemokine specific neutralizing antibodies was examined. This lavage fluid was highly chemotactic for freshly isolated human neutrophils. Chemotaxis of isolated human neutrophils was also measured in response to human BAL fluid (described above) with or without 10 μg/ml anti-human CCL7 neutralizing antibody (anti-human CCL7 AF-282-NA, R7D Systems) or 10 μg/ml anti-human IL-8 neutralizing antibody (anti-human IL-8 AB-208-NA, R&D Systems). BAL fluid was incubated for 10 min with each neutralizing antibody prior to the addition of 5×104 neutrophils per well. Neutralisation of CXCL8 with specific antibody decreased neutrophil chemotaxis in response to lavage fluid obtained from LPS-challenged volunteers (FIG. 17c). Importantly, neutralisation of CCL7 also significantly decreased neutrophil chemotaxis (p=0.036) and was more effective at preventing neutrophil chemotaxis than CXCL8 neutralisation (p=0.017). A combination of neutralizing antibodies against CXCL8 and CCL7 significantly reduced neutrophil chemotaxis (p=0.015) to the same degree as CCL7 alone, thereby demonstrating the functional importance of CCL7.

The levels of CCL7 and CCL2 in BAL fluid obtained from patients with a confirmed diagnosis of ALI within an intensive care unit environment were then measured. CCL7 and CCL2 levels were significantly increased in BAL fluid obtained from patients with ALI compared to healthy individuals (FIGS. 17d and 17f), suggesting that CCL7 and CCL2 may play an important role during ALFARDS. CCL7 levels were 10-fold higher in the BAL fluid of ALI patients (87.7+/−17.7 μg/ml) compared to the levels in BALF from LPS-challenged volunteers (8.6+/−2.1 μg/ml), indicating that CCL7 levels may be associated with disease severity. As observed with BAL fluid obtained from LPS-challenged volunteers, the BAL fluid from ALI patients was highly chemotactic for neutrophils. Surprisingly, neutralisation of CXCL8 alone did not significantly reduce neutrophil chemotaxis in response to all of the ALI BAL fluid (p=0.09) (FIG. 17e), demonstrating that other neutrophil chemotactic mediators may be present within the BALF. However, neutralisation of CCL7 significantly reduced (p=0.007) human neutrophil chemotaxis in response to this BAL fluid, while neutralisation of both CXCL8 and CCL7 reduced chemotaxis further (p=0.0007). Taken together these data confirm the functional importance of CCL7 and CCL2 in modulating human neutrophil chemotaxis in the setting of ALI and suggest that blockade of CXCL8 alone may not be sufficient to block neutrophil recruitment in this disease setting.

### 6. Neutrophils Express CC-Chemokine Receptors in the Inflamed Lung

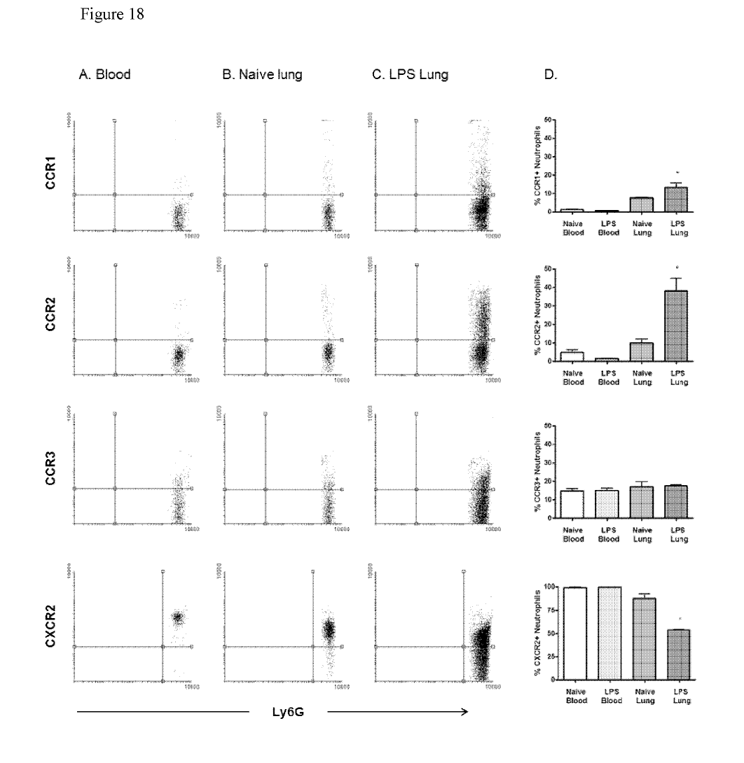

In order to assess the capacity of neutrophils to respond to CC-chemokines, the expression of the known CC-chemokine receptors CCR1, CCR2 and CCR3 on neutrophils isolated from the blood, naïve lung and LPS-challenged lung was assessed, and compared with the major neutrophil chemoattractant CXCL1 (KC) receptor CXCR2 by flow cytometry. Minimal expression of CCR1 or CCR2 was observed on neutrophils isolated from the blood (FIG. 18A), while only a small percentage of blood neutrophils expressed CCR3 (only flow cytometry plots of blood from LPS-challenged animals are shown for purposes of clarity, as LPS challenge did not affect CRR expression on blood neutrophils). In comparison, nearly all neutrophils isolated from the blood expressed CXCR2 (FIG. 18A, D). A small percentage of neutrophils isolated from naïve lung expressed CCR1, CCR2 and CCR3 (FIG. 18B), compared to >95% of neutrophils that expressed CXCR2 (FIG. 18B, D). However, a significantly higher percentage of neutrophils isolated from lung tissue following LPS challenge expressed CCR1 and particularly CCR2 (FIG. 18C, D). The percentage of neutrophils expressing CCR2 was only ˜10% in naïve lung compared to greater than 35% following LPS challenge (FIG. 18D). There was no percentage increase in neutrophils expressing CCR3 isolated from inflamed lung tissue (FIG. 18D). Interestingly, the percentage of CXCR2 expressing neutrophils isolated from inflamed lung tissue (>95%) decreased following challenge with LPS compared to naïve lung (<60%) (FIG. 18D), indicating that neutrophils acquire novel chemokine receptors when migrating into lung tissue during an inflammatory response.

1. An antagonist of:
(a) CCL7, PAR₁ or another member of the PAR₁-CCL7 axis; or
(b) CCL2;
for use in the treatment or prevention of acute inflammation associated with the accumulation of neutrophils in the respiratory tract.

2. An antagonist according to claim 1 wherein the other member of the PAR₁-CCL7 axis is CCR1, CCR2 or CCR3.

3. An antagonist according to claim 1 or 2 for use in the treatment or prevention of acute inflammation of neutrophils within the lung airspaces, bronchi, bronchial wall or interstitial space.

4. An antagonist according to any one of the preceding claims for use in the treatment or prevention of acute lung injury (ALI) or acute respiratory distress syndrome (ARDS).

5. An antagonist according to claim 4 for use in the treatment or prevention of ALI or ARDS arising from direct or indirect causes.

6. An antagonist according to claim 5 for use in the treatment or prevention of ALI or ARDS arising from a direct cause selected from trauma to the lung, bacterial or viral infection or another respiratory disease; or from an indirect cause selected from sepsis, pancreatitis and tissue trauma distal to the lung.

7. An antagonist according to claim 6 wherein the other respiratory disease is infant respiratory distress syndrome (IRDS), bronchiestasi or chronic obstructive pulmonary disease (COPD).

8. An antagonist according to any one of the preceding claims, wherein said antagonist blocks the interaction between:
(a) CCL7 and CCR1;
(b) CCL7 and CCR2;
(c) CCL7 and CCR3;
(d) CCL2 and CCR1;
(e) CCL2 and CCR2; or
(f) CCL2 and CCR3.

9. An antagonist according to any one of the preceding claims, which comprises an antibody, a double-stranded RNA, an antisense RNA, an aptamer, or a peptide or peptidomimetic that blocks the function of its target, wherein said target is a member of the PAR₁-CCL7 axis, or CCL2.

10. An antagonist antibody according to claim 9, which is a monoclonal antibody.

11. An antagonist monoclonal antibody according to claim 10, which is an an antibody to CCL7 or CCL2.

12. An antibody according to claim 11 which is an antibody to CCL7 whose epitope is located in the N-terminal region of CCL7, in the N-loop of CCL7, in the 30 s-loop of CCL7, adjacent to a disulfide bond in CCL7, in the alpha helix region of CCL7.

13. An antagonist according to any one of the preceding claims for use in the treatment or prevention of acute inflammation associated with the accumulation of neutrophils in the respiratory tract by down-regulating neutrophil recruitment and/or neutrophil accumulation.

14. An antagonist according to any one of the preceding claims, wherein the effect of the antagonist is by inhibiting neutrophil migration.

15. An antagonist according to any one of the preceding claims which is for intranasal or inhalational administration.

16. An antagonist according to any one of the preceding claims, for use in combination with one or more additional agents for the treatment and/or prevention of acute inflammation associated with the accumulation of neutrophils in the respiratory tract.

17. An antagonist for use according to claim 16, wherein said additional agent is an antagonist of CXCL8.

18. An antagonist for use according to claim 17, wherein said additional agent is an anti-CXCL8 antibody.

19. Use of an antagonist of CCL7, PAR₁, another member of the PAR₁-CCL7 axis, or CCL2 in the manufacture of a medicament for the treatment or prevention of acute inflammation associated with the accumulation of neutrophils in the respiratory tract.

20. A method of treating or preventing acute inflammation associated with the accumulation of neutrophils in the respiratory tract comprising administering to a patient in need thereof an effective amount of an antagonist of CCL7, PAR₁, another member of the PAR₁-CCL7 axis, or CCL2.
